(19) United States
(12) Patent Application Publication
(10) Pub. No.: US 2025/0197882 A1
(43) Pub. Date: Jun. 19, 2025

(54) USE OF JAZ PROTEIN AND ITS DERIVATIVES IN AGRICULTURAL PEST PREVENTION AND CONTROL

(71) Applicant: INSTITUTE OF COTTON RESEARCH OF THE CHINESE ACADEMY OF AGRICULTURAL SCIENCES, Anyang City (CN)

(72) Inventors: Fuguang LI, Anyang City (CN); Maozhi REN, Anyang City (CN); HuiJuan MO, Anyang City (CN)

(21) Appl. No.: 18/847,958

(22) PCT Filed: Mar. 22, 2022
(86) PCT No.: PCT/CN2022/082179

§ 371 Date: Sep. 17, 2024

(30) Foreign Application Priority Data
Mar. 17, 2022 (CN) 202210263097.X

(51) Int. Cl. C12N 15/82; A01N 63/50; A01P 7/04

(52) U.S. Cl. CPC C12N 15/8286; A01N 63/50; A01P 7/04; C12N 15/8205

(57) ABSTRACT

The present invention discloses a use of JAZ protein and derivatives thereof in prevention and control of agricultural pest. The present invention provides a use of a GhJAZ protein comprising a NGR domain, or coding nucleic acid thereof, or a recombinant vector, an expression cassette, or a recombinant bacterium, or a substance having the GhJAZ protein as an active ingredient in at least one of: 1) killing insect; 2) insect resistance; 3) preparation of insecticidal or insect resistant products; 4) improvement of plant insect resistance; 5) cultivation of insect resistant plants. The present invention discovered for the first time that a JAZ protein, especially NGR-JAZ and derived polypeptides thereof, have broad-spectrum insect resistance, for which no relevant reports are found yet all over the world, and it has significant application value in major agricultural pest control.

### RELATED APPLICATIONS

The present application is a U.S. National Phase of International Application Number PCT/CN2022/082179 filed on Mar. 22, 2022, which claims priority to Chinese Application Number 202210263097.X filed on Mar. 17, 2022.

### INCORPORATION BY REFERENCE

The sequence listing provided in the file entitled SQL_REV_GPB_2.txt which is an ASCII text file that was created on Sep. 16, 2024, and which comprises 47,585 bytes, is hereby incorporated by reference in its entirety.

### TECHNICAL FIELD

The present invention belongs to the field of plant genetic engineering and biological control technology, and particularly relates to use of JAZ protein and derivatives thereof in agricultural pest prevention and control.

### BACKGROUND ART

Insect pests are the main factor causing crop losses worldwide, and have characteristics of a wide variety of species, significant impact, and frequent outbreaks of disasters. The scope and severity of their occurrence often result in significant losses to China's national economy, especially agricultural production. Among them, fall armyworm (Spodoptera frugiperda), cotton bollworm (Helicoverpa armigera), fleahopper, corn borer, cotton aphid, wheat aphid, etc. have become major agricultural pests that seriously affect agricultural production around the world.

The fall armyworm, Spodoptera frugiperda (J. E. Smith) in Latin name, belongs to the Lepidoptera order and Noctuidae family, becomes a major agricultural pest globally warned by the Food and Agriculture Organization of the United Nations. In January 2019, the Spodoptera frugiperda was introduced from Myanmar and spread northward in Yunnan Province, China, becoming a major migratory pest with an annual cycle of “northward migration and southward return”. It can feed on plants of over 350 species from more than 76 families, causing serious economic losses and posing a serious threat to food security around the world. For a long time, the control of Spodoptera frugiperda has relied on chemical control and Bt transgene crops, but recent research has shown that Spodoptera frugiperda has developed resistance to various pesticides and Bt transgenic corn.

The cotton bollworm, Helicoverpa armigera (Hubner) in Latin name, belongs to the Lepidoptera order and Noctuidae family. It is widely distributed in China and around the world, with over 200 species from more than 30 families as host plants. It occurs in cotton and vegetable planting areas in China. Helicoverpa armigera is an important pest on crops such as cotton, peanuts, and soybeans, and can also harm wheat, corn, sorghum, beans, tobacco, sesame, sunflowers, apples, pears, peaches, grapes, etc.

Traditionally, the main method for controlling insect and pest populations is to apply broad-spectrum chemical insecticides. For a long time, spraying large amounts of chemical insecticides not only enhances pest resistance and damages beneficial insects and other ecological systems, but also seriously pollutes the environment, increases production costs, and breaks ecological balance. Therefore, reducing the use of insecticides, carrying out emergency prevention and control, and developing new green biological prevention and control methods, will be a very important task at present.

Cotton is one of the most important economic crops in the world, and its fiber is an important raw material for the textile industry. As one of the four major cotton production countries in the world, China's cotton planting area and output have always been among the top in the world. A plant hormone, jasmonic acid (JA), plays an important role in plant defense against pests by regulating the expression of downstream genes upon the attack of pest. JAZ protein, as a key regulatory factor of jasmonic acid signaling, is an important transcriptional repressor, mainly inhibits the transcriptional expression of downstream genes in jasmonic acid response, and. However, no reports on use of a plant transcription inhibitor JAZ protein in pest control are found to date.

NGR (Asn-Gly-Arg) is a tripeptide motif which is selected out via phage display technology and can specifically bind to tumor neovascularization. It can specifically bind to neovascularization through an aminopeptidase N (also known as APN/CD13) on endothelial cells. Aminopeptidase N (APN/CD13) is a type II metalloexopeptidase that depends on zinc ions and belongs to the M1 aminopeptidase family, the physiological function of which is to participate in the degradation of N-terminal amino acids of substrate proteins.

In 1996, the first transgenic insect resistant plant in the world was approved for application in the United States. Over the past two decades, commercialization has proven that genetically modified crops have brought agricultural, environmental, economic, health, and social benefits to the world. However, they usually only have resistance to a narrower range of economically important pests, and some insects have developed resistance to certain insecticidal peptides in these genetically engineered crops.

Therefore, there remains a need for new insecticidal proteins with a wider range of insecticidal activity against insect pests, such as toxins with activity against more species from Lepidoptera, Homoptera, Hemiptera, etc., to provide new genetic sources for genetically modified crops and engineered strains. In addition, there is still a need for biopesticides with improved insecticidal activity and activity against various insects that have developed resistance to existing insecticides and insecticidal proteins, which have important economic, social, and ecological benefits.

### SUMMARY OF THE INVENTION

One objective of the present invention is to provide the following applications.

The present invention provides a use of a GhJAZ protein comprising a NGR domain or its coding nucleic acid, or a recombinant vector, expression cassette or recombinant bacterium comprising the coding nucleic acid, or a substance comprising the GhJAZ protein as an active ingredient in at least one of:
1) insecticidal;
2) insect resistance;
3) preparation of insecticidal or insect resistant products;
4) improvement of plant insect resistance;
5) cultivation of insect resistant plants;
6) prevention and control of plant diseases and pests;
the amino acid sequence of the NGR domain is SEQ ID NO: 17.

The GhJAZ protein is derived from Gossypium, Tripterygium wilfordii, Hibiscus syriacus, Durio zibethinus, Herrania umbratica, Theobroma cacao, Nelumbo nucifera, and durian (Durio zibethinus).

In the above use, the GhJAZ protein comprising the NGR domain is any of:
1) a protein shown in SEQ ID NO: 5, SEQ ID NO: 7, SEQ ID NO: 9, SEQ ID NO: 11, SEQ ID NO: 13, or SEQ ID NO: 15;
2) a protein derived from A by adding to an end of the amino acid sequence of the protein shown in 1) a tag sequence and having the same activity;
3) the protein is a protein that has at least 95%, 98%, and 99% sequence identity with that of 1) and comprises a polypeptide shown in SEQ ID NO: 17.

In the above use, the coding nucleic acid is a DNA molecule of any of 1)-3):
1) a DNA molecule having a coding region shown in SEQ ID NO: 6, SEQ ID NO: 8, SEQ ID NO: 10, SEQ ID NO: 12, SEQ ID NO: 14, or SEQ ID NO: 16;
2) a DNA molecule that is capable of hybridizing with the defined DNA sequence shown in 1) under strict condition and encodes a protein having the same function;
3) a DNA sequence that has at least 70%, at least 75%, at least 80%, at least 85%, at least 90%, at least 95%, at least 96%, at least 97%, at least 98%, or at least 99% homology with the defined DNA sequence shown in 1) and encodes a protein having the same function.

In the above use, the insect is a plant pest;
and/or, the plant pest is a Lepidoptera pest.

In the above use, the recombinant vector is a vector for expressing a nucleic acid encoding a GhJAZ protein comprising a NGR domain. In the embodiments of the present invention, the pET-30a-GhJAZ8 vector or pMDC100-GhJAZ8 vector is used as an example. In the above use, the substance having a GhJAZ protein as an active ingredient is a fusion protein formed by fusing the GhJAZ protein with other protein(s) and having the GhJAZ protein as the active ingredient, and a mixture composed of the GhJAZ protein and other components and having the GhJAZ protein as the active ingredient.

Another objective of the present invention is to provide a method for cultivating an insect resistant transgenic plant.

The method provided by the present invention is 1) or 2):
1) the method comprises the steps of: increasing the content and/or activity of a GhJAZ protein comprising a NGR domain in a target plant to obtain a transgenic plant;
2) the method comprises the steps of: increasing the expression of a nucleic acid molecule encoding a GhJAZ protein comprising a NGR domain in a target plant to obtain a transgenic plant; the amino acid sequence of the NGR domain is SEQ ID NO: 17;
the insect resistance of the transgenic plant is higher than that of the target plant.

In the above method, the insect is a plant pest;
and/or, the plant pest is a Lepidoptera pest.

The above GhJAZ protein comprising the NGR domain can be any of:
1) a protein shown in SEQ ID NO: 5, SEQ ID NO: 7, SEQ ID NO: 9, SEQ ID NO: 11, SEQ ID NO: 13, or SEQ ID NO: 15;
2) a protein derived from A by adding to an end of the amino acid sequence of the protein shown in 1) a tag sequence and having the same activity;
3) the protein is a protein that has at least 95%, 98%, and 99% sequence identity with 1) and comprises a polypeptide shown in SEQ ID NO: 17.

The above nucleic acid molecule encoding the GhJAZ protein comprising the NGR domain is a DNA molecule of any of 1)-3):
1) a DNA molecule having a coding region shown in SEQ ID NO: 6, SEQ ID NO: 8, SEQ ID NO: 10, SEQ ID NO: 12, SEQ ID NO: 14, or SEQ ID NO: 16;
2) a DNA molecule that hybridizes with the defined DNA sequence shown in 1) under strict conditions and encodes a protein having the same function;
3) a DNA molecule that has at least 70%, at least 75%, at least 80%, at least 85%, at least 90%, at least 95%, at least 96%, at least 97%, at least 98%, or at least 99% homology with the defined DNA sequence shown in 1) and encodes a protein having the same function.

Another objective of the present invention is to provide a method for killing insects or resisting insects, comprising the steps of: applying a GhJAZ protein comprising a NGR domain to the insects, to achieve insecticidal or insect resistance.

The insect is a plant pest, specifically a Lepidoptera pest.

Use of a substance that inhibits the expression of a NGR domain in a GhJAZ protein comprising the NGR domain in reducing the insecticidal or insect resistance of the GhJAZ proteins comprising the NGR domain is also within the scope of protection of the present invention; or, use of a substance that mutates a NGR domains in a GhJAZ protein comprising the NGR domains in reducing the insecticidal or insect resistance of the GhJAZ protein containing the NGR domain is also within the scope of protection of the present invention.

The above substance that inhibits the expression of a NGR domain in a GhJAZ protein comprising the NGR domain is to mutate the N at position 43 to L and the R at position 45 to K in the NGR domain. In the examples of the present invention, GhJAZ8 is mutated to GhJAZ8^{LGK}.

The above-mentioned insects are Lepidoptera pests; in the examples of the present invention, Lepidoptera pests are exemplified as fall armyworm (Spodoptera frugiperda), cotton bollworm (Helicoverpa armigera), fleahoppers, and aphids, and may also be Spodoptera litura, Helicoverpa armigera, Trichoplusia ni, Hyponomoma kahamanoa, Galleria mellonella, Maniola hyperantus, Aricia agestis, Manduca sexta, Brenthis ino, Arctia plantaginis, Chilo suppressalis, Paragge aegeria, Vanessa tameamea, Bicycles anynana, Amyelois transitella, Melitaea cinxia, Leptidea sinapis, Pieris rapae, or Pieris brassicae.

In the present invention, the plant is maize, sorghum, wheat, sunflowers, tomatoes, cruciferous species, Capsicum species, potatoes, cotton, rice, soybeans, Beta vulgaris L., sugarcane, tobacco, or barley.

In one aspect, the present invention provides a JAZ and a derivative thereof (including a polypeptide) that imparts insecticidal activity to bacteria, plants, plant cells, tissues, and seeds, as well as a composition and a method of a modified product based on the above substance. The JAZ and the derivative thereof (including a polypeptide) and the composition of the modified product based on the above substance comprise a nucleic acid molecule encoding a sequence of a pesticidal and an insecticidal polypeptide, a vector containing the nucleic acid molecule, as well as a host cell containing the vector. The JAZ and the derivative thereof (including a polypeptide) and the composition of the modified product based on the above substance further comprise pesticidal polypeptide sequences and antibodies of the peptides. Nucleic acid sequences can be used in DNA constructs or expression cassettes for transformation and expression in organisms, including microorganisms, plants, and animals. Nucleotide or amino acid sequences can be synthetic sequences designed for expression in organisms, including but not limited to microorganisms, plants, and animals. The compositions further comprise transformed plants, plant cells, tissues, seeds, animals, and microorganisms.

Specifically, provided are isolated or recombinant nucleic acid molecules encoding a JAZ and a derivative thereof (including a polypeptide) and a modified product (including an amino acid substitution, a deletion, an insertion) based on the above substances. In addition, nucleotide sequences corresponding to Asn-Gly-Arg peptide are covered. Nucleic acid sequences complementary or hybridized to the nucleic acid sequences of the embodiments are also covered.

In a specific embodiment of the present invention, a nucleic acid optimized for increased expression in a host organism can be used to produce a transformed biomaterial. For example, a JAZ and a derivative thereof (including a polypeptide) and one of the modified products based on the above substance in the embodiments of the present invention can be reverse translated to generate nucleic acids comprising codon(s) optimized for expression in a specific host, such as maize (Zea mays), rice (Oryza sativa), cotton (Gossypium hirsutum) and other receptor plants. The expression of coding sequences in such transformed plants (such as dicotyledonous or monocotyledonous plants) will result in production of insecticidal JAZ and the derivatives thereof (comprising polypeptides) and modified products based on the aforementioned substances, and confer increased insect resistance to the organism.

In another aspect, the present invention provides a method for producing peptides and using those peptides to prevent and control or kill major agricultural pests. The transgenic organism of the embodiment expresses one or more of the pesticidal sequences disclosed herein. In various embodiments, the transgenic organism further comprises one or more additional genes having insect resistance, such as one or more additional genes for preventing and controlling Lepidoptera, Homoptera, and Hemiptera pests. The skilled in the art should understand that the transgenic organism may contain any gene that confers the agronomic trait of interest.

### BRIEF DESCRIPTION OF THE DRAWINGS

FIGS. 1A-1C shows protein expression of GhJAZ5, GhJAZ8, and GhJAZ16 analyzed by Western blot; the size of GhJAZ5 (FIG. 1A) protein is 27.28 kDa, GhJAZ8 (FIG. 1B) protein is 14.7 kDa, and GhJAZ16 (FIG. 1C) protein is 40.7 kDa.

FIG. 2 shows cytotoxicities of protein gradient concentration solutions of GhJAZ5, GhJAZ8, and GhJAZ16 on the Sf9 ovarian cell line of Spodoptera frugiperda.

FIG. 3 shows an amino acid sequence alignment between NGR GhJAZs and JAZ protein family members in upland cotton (Gossypium hirsutum).

FIGS. 4A-4C shows the interaction between NGR GhJAZs and SfAPN4; FIG. 4A illustrates the N-terminal signal peptide, GAMEN motif, HEX2HX18E motif, and GPI anchor signal functional conserved domains of SfAPN4; FIG. 4B, Yeast two-hybrid Y2H experiment shows the interaction between GhJAZ protein containing a NGR domain and SfAPN4 through the NGR; FIG. 4C, co immunoprecipitation CoIP (Co immunoprecipitation) experiment shows the interaction between GhJAZ8 containing a NGR domain and SfAPN4.

FIG. 5 shows the amino acid alignment of SfAPN4 (SEQ ID NO: 21) and APN4 in the homologs of the following species.

FIGS. 6A-6F shows the bioassay of GhJAZ8 protein against major agricultural pests such as Spodoptera frugiperda and Helicoverpa armigera; FIGS. 6A and 6B show the SDS-PAGE (left figure) and Western blot (right figure) results of purified GhJAZ8 and GhJAZ8^{LGK} proteins;

FIGS. 6C and 6D show the growth inhibitory phenotypes of GhJAZ8 protein and GhJAZ8^{LGK} protein with mutated “NGR” domain against Spodoptera frugiperda and Helicoverpa armigera;

FIGS. 6E and 6F show the growth inhibitory rate data of GhJAZ8 protein and GhJAZ8^{LGK} protein with mutated “NGR” domain against Spodoptera frugiperda and Helicoverpa armigera.

FIG. 7 shows the determination of apoptosis in Sf9 cells of Spodoptera frugiperda by GhJAZ13, GhJAZ14, GhJAZ24, GhJAZ27, and GhJAZ28.

FIG. 8 shows the in vivo interaction between GhJAZ8 protein and SfHDAC3 protein by the CoIP experiment.

FIG. 9 shows the amino acid sequence homology results between SfHDAC3 (SEQ ID NO: 23) and that of other species of pests, with the same amino acids marked below the sequence.

FIG. 10 shows the structural diagram of the plant expression vector pMDC100-GhJAZ8.

FIG. 11 shows the PCR identification of GhGNPBY22005JAZ8 transgenic rice, maize, cotton, and tobacco.

FIG. 12 shows the phenotypes of GhJAZ8 transgenic rice and wild-type rice on the 7th day after being fed by Spodoptera frugiperda.

FIG. 13 shows the corrected mortality rate of Cnaphalocrocis medinalis after feeding on GhJAZ8 transgenic rice.

FIG. 14 shows the midgut tissue slices of Helicoverpa armigera larvae after feeding on GhJAZ8 transgenic cotton, the midgut tissue structure of the Helicoverpa armigera was stained with TUNNEL-IF; +WT: phenotype of midgut tissue of the Helicoverpa armigera after feeding on wild-type cotton leaves; +OE: phenotype of midgut tissue of the Helicoverpa armigera after feeding on GhJAZ8 transgenic cotton leaves.

FIG. 15 shows the midgut tissue slices of Spodoptera frugiperda larvae after feeding on GhJAZ8 transgenic maize, the midgut tissue structure of the Spodoptera frugiperda was stained with TUNNEL; +WT: phenotype of midgut tissue of Spodoptera frugiperda after feeding on wild-type maize leaves; +OE: phenotype of midgut tissue of Spodoptera frugiperda after feeding on GhJAZ8 transgenic maize leaves.

FIGS. 16A and 16B shows the midgut tissue sections of Spodoptera frugiperda larvae after feeding on GhJAZ8 transgenic tobacco. The midgut tissue structure of Spodoptera frugiperda was stained with HE (40×).

### DETAILED DESCRIPTION OF EXAMPLES

The experimental methods used in the following examples are conventional methods unless otherwise specified.

The materials, reagents, etc. used in the following examples can be obtained from commercial sources unless otherwise specified.

The PBS used in the following examples is from Shenggong, product number E607016-0500, 1×PBS, pH 7.2 was used.

Fall armyworm Spodoptera frugiperda, source: Institute of Plant Protection, Henan Academy of Agricultural Sciences, recorded in the following literature, Gui F, Lan T, Zhao Y, Guo W, Dong Y, Fang D, Liu H, Li H, Wang H, Hao R, Cheng X, Li Y, Yang P, Sahu S K, Chen Y, Cheng L, He S, Liu P, Fan G, Lu H, Hu G, Dong W, Chen B, Jiang Y, Zhang Y, Xu H, Lin F, Slippers B, Postma A, Jackson M, Abate B A, Tesfaye K, Demie A L, Bayeleygne M D, Degefu D T, Chen F, Kuria P K, Kinyua Z M, Liu T X, Yang H, Huang F, Liu X, Sheng J, Kang L. Genomic and transcriptomic analysis unveils population evolution and development of pesticide resistance in fall armyworm Spodoptera frugiperda. Protein Cell. 2020 Oct. 27. doi: 10.1007/s13238-020-00795-7. Epub ahead of print. PMID: 33108584. The name of this material in the literature is fall armyworm.

Source of cotton bollworm Helicoverpa armigera: Cotton Research Institute, Chinese Academy of Agricultural Sciences, recorded in the following literature, Zhang H, Tian W, Zhao J, Jin L, Yang J, Liu C, Yang Y, Wu S, Wu K, Cui J, Tabashnik B E, Wu Y Diverse genetic basis of field-evolved resistance to Bt cotton in cotton bollworm from China. Proc Natl Acad Sci USA. 2012 Jun. 26; 109(26):10275-80. doi: 10.1073/pnas.1200156109. Epub 2012 Jun. 11. PMID: 22689968; PMCID: PMC3387040. The name of this material in the literature is cotton Bollworm.

The following examples are provided in an illustrative manner rather than in a restrictive manner. Below, the technical solution of the present invention will be clearly and completely described in conjunction with the Examples of the present invention. Obviously, the described Examples are only a part of the embodiments of the present invention, and not all of embodiments. Based on the Examples of the present invention, all other embodiments obtained by ordinary skilled persons in the art without creative labor are within the scope of protection of the present invention.

### Example 1: Discovery and Cloning of JAZ Protein in Cotton

I. Toxicity Analysis of Cotton JAZ Protein on Spodoptera frugiperda Cell Line Sf9

### 1. Cotton GhJAZ5 (Gh_A05G0260.1), GhJAZ8 (Gh_A05G1241.1), GhJAZ16 (Gh_A12G2441.1)

JAZ is one of the typical plant derived transcriptional repressor proteins. Based on the sequenced whole genome database of Gossypium Linn. (upland cotton Gossypium hirsutum), previous studies have reported that there are 30 JAZ members in the JAZ family of upland cotton (Sun H, Chen L, Li J, Hu M, Ullah A, He X, Yang X, Zhang X. The JASMONATE ZIM-Domain Gene Family Mediates JA Signaling and Stress Response in Cotton. Plant Cell Physiol. 2017 Dec. 1; 58(12):2139-2154. doi: 10.1093/pcp/pcx148. PMID: 29036515). Three representative cotton JAZs: GhJAZ5(Gh_A05G0260.1), GhJAZ8(Gh_A05G1241.1), GhJAZ16(Gh_A12G2441.1) are selected (Gene ID represents gene accession number in the CottonFGD database, https://cottonfgd.org/).

### 2. Prokaryotic Protein Expression and Purification of GhJAZ5, GhJAZ8, and GhJAZ16

pET-30a-GhJAZ5: the coding gene DNA sequence of GhJAZ5 shown in SEQ ID No. 2 was inserted into a pET-30a (+) vector (Ubao Biotech, item number: VT1212) between NdeI and HindIII to obtain a vector expressing GhJAZ5 protein (SEQ ID NO: 1);
PET-30a-GhJAZ8: the coding gene DNA sequence of GhJAZ8 shown in SEQ ID No. 4 was inserted into a pET-30a (+) vector (Ubao Biotech, item number: VT1212) between NdeI and HindIII to obtain a vector expressing GhJAZ8 protein (SEQ ID NO: 3);
PET-30a-GhJAZ16: the coding gene DNA sequence of GhJAZ16 shown in SEQ ID No. 6 was inserted into a pET-30a (+) vector (Ubao Biotech, item number: VT1212) between NdeI and HindIII to obtain a vector expressing GhJAZ16 protein (SEQ ID NO: 5);
Escherichia coli BL21 was transformed with above pET-30a-GhJAZ5, pET-30a-GhJAZ8, and pET-30a-GhJAZ16, respectively, placed on ice for 30 minutes, followed by a heat shock at 42° C. for 60 seconds, and a stand on ice for 2 minutes, as well as addition of LB medium, the mix was cultured at 37° C., 200 rpm, for 1 hour, then coated on LB plates with corresponding antibiotics and cultured at 37° C. Single clones were selected after occurrence of the clonal plaques, and cultured with shaking, then the culture was expanded until the medium began to turn slightly white. Then IPTG (specifically 0.5 mM final concentration) was added for induction and temperature of 37° C. was set for overnight culture. Then the resulted culture was centrifuged at 5000 rpm, 4° C. for 10 minutes, and pellet was collected and resuspended in equal volume PBS (with PMSF and DTT added). The suspension was sonicated (sonication time: 20 minutes, interval time: 5 seconds, sonication power: 200 W) for 5 seconds, and placed on ice for 5 seconds, until the resuspended solution becomes significantly clear. A further centrifugation at 13000 rpm, 4° C., for 10 minutes was conducted to separate the supernatant and inclusion bodies.

The supernatant was transferred into a protein purification column (Genscript Biotech, item number: L00250-25; elution with low pH buffer and imidazole solution), washed and eluted after being incubated, and the eluent was collected as a target protein solution (Elution buffer (1 liter): 50 mM NaH₂PO₄, 300 mM NaCl, 250 mM imidazole), Adjust pH to 8.0 using NaOH).

Detection was conducted using a protein pre-staining solution and SDS-PAGE, a protein solution was collected.

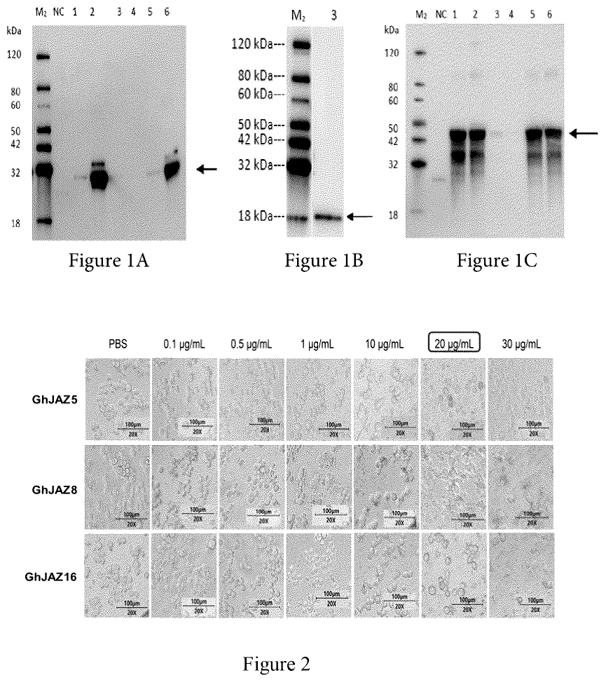

The Western blot analysis results were shown in FIGS. 1A-1C, where samples 1-6 in FIG. 1A and FIG. 1C are: lane 1: lysate of cells induced at 15° C. for 16 hours; lane 2: lysate of cells induced at 37° C. for 4 hours; lane 3: the supernatant lysate of cells induced at 15° C. for 16 hours; lane 4: supernatant lysate of cells induced at 37° C. for 4 hours; lane 5: lysate debris of cells induced at 15° C. for 16 hours; lane 6: lysate debris of cells induced at 37° C. for 4 hours. It can be seen that the target protein could be obtained from the supernatant lysate of cells induced at 37° C. for 4 hours, with protein sizes of: GhJAZ5 (A) 27.28 kDa, GhJAZ8 (B) 14.7 kDa, and GhJAZ16 (C) 40.7 kDa.

3. Cell Toxicity of GhJAZ5, GhJAZ8, and GhJAZ16 on Sf9 Ovarian Cell Line of Spodoptera frugiperda

Using insect cell lines toxicity testing and mechanism of action research of insecticides, as a new detection method, has the advantages of good target homogeneity, easy control of experimental conditions, and low screening costs compared to traditional bioassay methods. It is suitable for toxicological research of insecticidally active substances. In recent years, scholars at home and abroad have conducted extensive research on the activity of plant natural active ingredients on insect cells, and have achieved many research results of the action mechanism of plant natural active ingredients on insect cells, showing a good correlation between experimental results and in vivo toxicity testing and field experiments.

Fluorescence staining experiment: toxic effect of GhJAZ protein on Sf9 cell line (CL-0205, Procell) was detected using a Calcein/PI Cell Viability and Cytotoxicity Detection kit (Catalogue No.: C2015L, Beyotime, China). The Calcein/PI Cell Activity and Cytotoxicity Detection Kit is a very convenient kit for detecting the viability of animal cells based on Calcein AM (Calcein AM) and PI (Propidium Iodide) dual fluorescence staining. The specific steps are as follows: Adherent cells were blown and transferred to a 15 mL EP tube, centrifuged at 860-870 rpm for 5 minutes. Remove the supernatant (to reduce cell debris), add fresh culture medium, and gently mix well with a pipette. After counting on a hemocytometer, cells were seeded into a 96 well plate with 5×10⁴ cells per well. After 24 hours of culture, different concentrations of GhJAZ protein solution (solvent: PBS) were added. After another 24 hours of culture, Calcein/PI cell viability and cytotoxicity detection kit was added. After 30 minutes of culture, fluorescence microscopy images were taken. The maximum excitation wavelength of Calcein AM hydrolyte Calcein is 494 nm, and the maximum emission wavelength is 517 nm; the maximum excitation wavelength of PI-DNA complex is 535 nm, and the maximum emission wavelength is 617 nm.

The cytotoxicity results of GhJAZ5, GhJAZ8, and GhJAZ16 protein solutions on an ovarian cell line Sf9 of Spodoptera frugiperda were shown in FIG. 2. As the concentrations of GhJAZ5, GhJAZ8, and GhJAZ16 protein solutions increased, Sf9 cells begin to lyse and a large amount of cell debris was produced, indicating that GhJAZ5, GhJAZ8, and GhJAZ16 protein solutions all exhibited certain cytotoxic effects on Sf9 cells. Among them, GhJAZ8 protein solution has the strongest toxicity to Sf9 cells.

Therefore, GhJAZ8 protein was selected for further study below.

### II. Identification of Cotton GhJAZ Gene Containing NGR Domain

Amino acid sequence of GhJAZ8 protein was aligned with that of members of JAZ protein family in a upland cotton (Gossypium hirsutum).

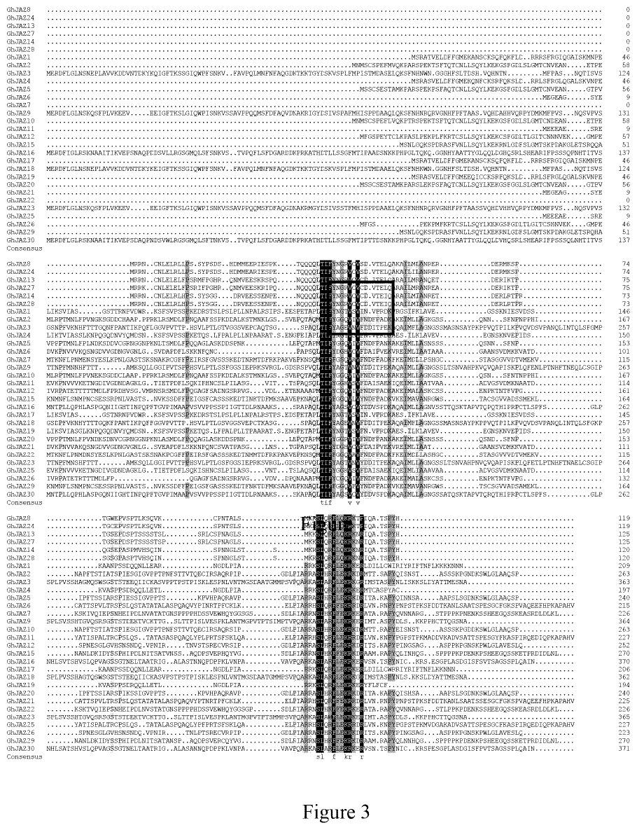

As shown in FIG. 3, it can be seen that GhJAZ8 has a NGR (Asn Gly Arg amino acid) domain. Further analysis revealed that among 30 JAZs in cotton, there are a total of 6 JAZs containing an NGR domain. The genome annotation naming and Gene IDs are: GhJAZ8(Gh_A05G1241.1, SEQ ID No. 3, SEQ ID No. 4), GhJAZ13 (Gh_A09G0741, SEQ ID No. 7, SEQ ID No. 8), GhJAZ14(Gh_A10G0388, SEQ ID No. 9, SEQ ID No. 10), GhJAZ24(Gh_D05G3842, SEQ ID No. 11, SEQ ID No. 12), GhJAZ27(Gh_D09G0743, SEQ ID No. 13, SEQ ID No. 14), GhJAZ28(Gh_D10G0403, SEQ ID No. 15, SEQ ID No. 16). It is predicted that six JAZ proteins containing a NGR domain should have similar activity and function. The core residue NGR (Asn43-Gly44-Arg45) that constitutes QQQQLTIFYNGRVCV (SEQ ID NO: 17, with an coding nucleic acid of SEQ ID NO: 18) is indicated with a frame line in the sequence.

Using BLAST and PSI-BLAST in the non redundant database (nr) of the National Center for Biotechnology Information (NCBI) in the United States, plant species were sequentially input in the “Organism” condition box, and a whole species genome BLAST was conducted at NCBI using the six most conserved JAZ amino acid sequences QQQQLTIFYNGRVCV (SEQ ID NO: 17) containing a NGR domain in a cotton, it was discovered that there is no QQQQLTIFYNGRVCV domain in the JAZ family in plants such as maize (Zea mays), rapeseed (Brassica napus), soybean (Glycine max), wheat (Triticum aestivum), rice (Oryza sativa), sweet potato (Ipomoea batatas), potato (Solanum tuberosum), broad bean (Vicia faba), pea (Pisum Sativum), mung bean (Phaseolus radiatus), Arabidopsis (Arabidopsis thaliana), tobacco (Nicotiana tabacum) etc.

Table 1 listed partial results of the whole species genome BLAST using QQQQLTIFYNGRVCV in NCBI, indicating the amino acid sequence alignment between QQQQLTIFYNGRVCV (SEQ ID NO: 17) and the following homologs.

TABLE 1
presents the partial results of whole species
genome BLAST using QQQQLTIFYNGRVCV in NCBI

| Species | Species | Protein Sequence |
|---|---|---|
| cotten | Gossypium | QQQQLTIFYNGRVCV |
| | | (SEQ ID NO: 17) |
| maize | Zea mays | QQLTIFYGGRVVV |
| | | (SEQ ID NO: 29) |
| rice | Oryza sativa | QLTIFYGGSVCV |
| | | (SEQ ID NO: 30) |
| rapeseed | Brassica napus | QRLTIFYNGKMCV |
| | | (SEQ ID NO: 31) |
| soybean | Glycine max | QQQPLTIFYDGKICV |
| | | (SEQ ID NO: 32) |
| wheat | Triticum | QQLTIFYGGRVVV |
| | aestivum | (SEQ ID NO: 33) |
| sweet | Ipomoea | QLTIFYSGKV |
| potato | batatas | (SEQ ID NO: 34) |
| potato | Solanum | EQQQLTIFYDGKVVV |
| | tuberosum | (SEQ ID NO: 35) |
| broad bean | Vicia faba | TIFY (SEQ ID NO: 36) |
| pea | Pisum sativum | LTIFY (SEQ ID NO: 37) |
| mung bean | Phaseolus | QQQPLTIFYDGKICV |
| | radiatus | (SEQ ID NO: 38) |
| arabidopsis | Arabidopsis | QLTIFYAGSVCV |
| | thaliana | (SEQ ID NO: 39) |
| tobacco | Nicotiana | QQQQLTIFYNGKVVV |
| | tabacum | (SEQ ID NO: 40) |

Similarly, using BLAST and PSI-BLAST in the non redundant database (nr) of the National Center for Biotechnology Information (NCBI) in the United States, without limiting species in the “Organism” condition box, a whole species genome BLAST at NCBI was conducted using the six most conserved JAZ amino acid sequences QQQQLTIFYNGRVCV containing a NGR domain in a cotton, it was discovered that the QQQQLTIFYNGRVCV domain is only present in the genome sequences of Gossypium, Tripterygium wilfordii, Hibiscus syriacus, Durio zibethnus, Herrania umbratica, Theobroma cacao, Nelumbo nucifera, Durio zibethinus, among which Tripterygium wilfordii, Hibiscus syriacus, Durio zibethnus and cotton etc are famous Chinese medicinal herbs.

Table 2 shows results of the whole species genome BLAST using QQQQLTIFYNGRVCV in NCBI, indicating that the amino acid sequence of QQQQLTIFYNGRVCV (SEQ ID NO: 17) is identical to that of homologs in the following species. The names of traditional Chinese medicine materials for homologs in the species have been listed.

Table 2 presents the results of whole species genome BLAST using QQQQLTIFYNGRVCV in NCBI

| | | Name of Traditional Chinese Medicine |
|---|---|---|
| Species | Species | Material |
| Gossypium | Gossypium | Kapok, Anemone Vitifolia, gossypetin, |
| | | gossypol |
| Tripterygium | Tripterygium | thunder god vine |
| wilfordii | wilfordii | |
| Hibiscus syriacus | Hibiscus | Bark, flower of hibiscus |
| Durio zibethinus | Salvia | salvia |
| Herrania | Mallow | Fructus Malvae Verticillatae |
| umbratica | | |
| Theobroma | Cacoa | Theobromine |
| cacao | | |
| Nelumbo | Nelumbo | Herba Andrographitis |
| nucifera | nucifera | |
| Durio zibethinus | Durian | |

### III. NGR Domain Guided Targeting of GhJAZ8 to SfAPN4

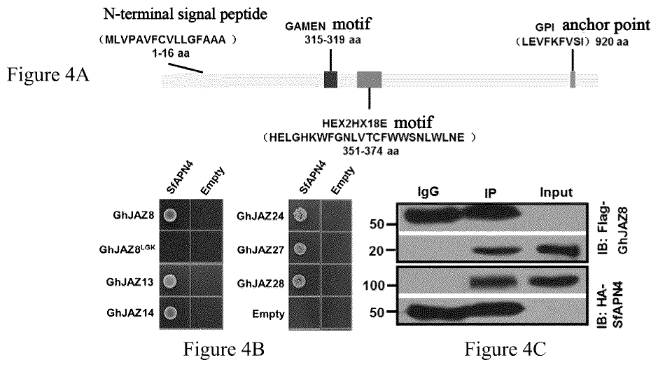

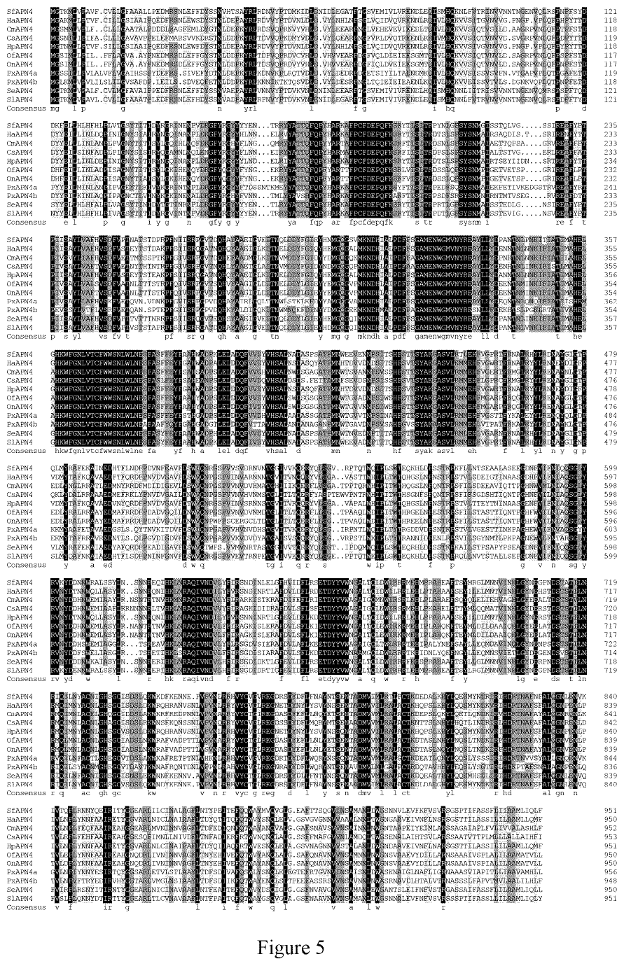

NGR is a recognized target peptide for cell surface receptor APN in medicine. Insect midgut APN is the earliest discovered functional receptor for Bt Cry1Ca toxin, and cotton GhJAZs containing NGR may enter insect midgut cells through APN. Due to the fact that APN receptor is a zinc dependent membrane-bound exopeptidase, based on the existing Spodoptera frugiperda transcriptome and genome databases in combination with the NCBI genome database, a BLAST alignment was performed using the amino acid sequence of human derived aminopeptidase N (APN/CD13, NCBI Reference Sequence: NM_001150. Submission date: PRI 23 Feb. 2022) to obtain an electronic sequence of Spodoptera frugiperda aminopeptidase N (SfAPN4) homologous to human derived aminopeptidase N. PCR amplification was performed via designed primers (template: maize type Spodoptera frugiperda larvae whole tissue cDNA, SfAPN4-F: 5′-CGCTGGGGCAACCATGGGTACCA-3′ (SEQ ID NO: 41), SfAPN4-R: 5′-GAAATACGAGACGACAACGACAT-3′ (SEQ ID NO: 42)). SfAPN4-R: 5′-GAAATACGACGACAACGACAT-3′ (SEQ ID NO: 42) to obtain a true sequence (the amino acid sequence of SfAPN4 is shown in SEQ ID NO: 21, and the nucleotide sequence of SfAPN4 is shown in SEQ ID NO: 22). BLAST alignment in NCBI revealed a high degree of homology with APN4 of Lepidoptera pests. Therefore, the Spodoptera frugiperda aminopeptidase N was named as SfAPN4. Domains and important sites of the protein are identified using Motif Scan (http://myhits.isb-sib.ch/cgi-bin/motif_scan) and InterproScan (http://www.ebi.ac.uk/interpro/search/sequence-search). SfAPN4 contains a N-terminal signal peptide, a GAMEN motif, a HEX2HX18E motif, and a GPI anchor signal functionally conserved domain typically conserved in a APN family (FIG. 4A).

### 1. Yeast Two-Hybrid Experiment

DNA fragments shown in PGADT7::GhJAZ8 (SEQ ID No. 3), PGADT7::GhJAZ8^{LGK} (SEQ ID No. 25), PGADT7::GhJAZ13 (SEQ ID No. 8), PGADT7::GhJAZ14 (SEQ ID No. 10), PGADT7::GhJAZ24 (SEQ ID No. 12), PGADT7::GhJAZ27 (SEQ ID No. 14) and PGADT7::GhJAZ28 (SEQ ID No. 16) were inserted between the BamH1 and EcoR1 of a PGADT7 plasmid (Unibio Biotech, VT1639), respectively, to obtain pGADT7+GhJAZ8, pGADT7+GhJAZ8^{LGK}, pGADT7+GhJAZ13, pGADT7+GhJAZ14, pGADT7+GhJAZ24, pGADT7+GhJAZ27 and pGADT7+GhJAZ28.

The DNA fragment shown in PGBKT7: SfAPN4 (SEQ ID NO: 22) was inserted between BamH1 and EcoR1 of a pGBKT7 plasmid (Unibio Biotech, catalog number: VT1638), to obtain a vector plasmid, named pGBKT7+SfAPN4.

PGADT7+different gene plasmids were cotransformed with pGBKT7+SfAPN4 plasmids into yeast competent Y2HGold cells (Shanghai Weidi Company, catalog number: YC1002), respectively, then Carrier DNA (95° C., 5 min) and PEG/LiAC were added and mixed well, followed by water incubation at 30° C. for 30 min (6-8 flips at 15 min for mixing well) and at 42° C. for 15 min (8 flips at 7.5 min for mixing well) and centrifugation at 5000 rpm for 40 seconds to discard the supernatant and the pellet resuspended in ddH2O. The mixture was centrifuged at 5000 rpm for 30 seconds to discard the supernatant, and the pellet was resuspended in ddH2O, coated on a double dropout medium (SD/-Leu/-Trp) and cultured at 28° C. for 2 days. After occurrence of the clonal plaques, the clones were selected and placed a quadruple dropout medium (SD Ade/-His/-Leu/-Trp, with Aba and X-α-gal added) and the interaction was observed after 4 days of culture at 28° C.

As shown in FIG. 4B, it can be seen that SfAPN4 could interact with GhJAZ8, GhJAZ13, GhJAZ14, GhJAZ24, GhJAZ27, and GhJAZ28 respectively, but loses interaction with GhJAZ8^{LGK} (N43G44R45 mutation to L43G44K45), indicating that the NGR domain guided GhJAZ8 targeting to SfAPN4.

### 2. Co-Immunoprecipitation (CoIP) Experiment

pcDNA3.1-GhJAZ8 vector: a vector obtained by constructing a GhJAZ8 flag tag (inserting a flag tag (GACTACAAAGACGATGACGACAAA (SEQ ID NO: 43) before T at position 358 of SEQ ID No. 4, i.e., adding the flag tag before the termination codon of the GhJAZ8 gene) into a pcDNA3.1 vector (Unibio Biotech, VT1001);
pcDNA3.1-SfAPN4 vector: obtained by constructing a SfAPN4-flag tag (inserting a HA tag (TACCCATACGATGTTCCAGATTACGCTTGA (SEQ ID NO: 44) before T at position 2857 of SEQ ID No. 22, i.e., adding a flag tag before the termination codon of the SfAPN4 gene) into a pcDNA3.1 vector (Unibio Biotech, VT1001);
the above two vectors were cotransfected into 293T cells (Procell, catalog number: CL-0005) to obtain cotransfected cells.

Verification of above cotransfected cells with IP-WB: add non-denaturing lysis buffer into a culture plate of the cotransfected cell and completely lyse the cells at 4° C. Centrifuge the lysate at 12000 rpm for 10 minutes and collect the supernatant. Add a corresponding antibody to the resultant non-denaturing protein solution and incubate it overnight at 4° C. Wash Pierce™ protein A/G agarose beads (Thermo Fisher Scientific, Inc.) with lysis buffer, add the pretreated beads to the cell lysate, and incubate at room temperature for 2 hours. Centrifuge the mixture at 2500 rpm for 5 minutes at 4° C. to remove the supernatant, and wash the agarose beads 5 times with 1 ml lysis buffer. Add an appropriate amount of protein loading buffer to the precipitate and incubate the sample in a 100° C. water bath for 10 minutes. Western blot was used to detect the target band. The results were shown in FIG. 4C, the in vivo interaction between SfAPN4 and GhJAZ8 was validated.

### IV. Sequence Conservation of SfAPN4 Among Pests

Using BLAST and PSI-BLAST, the orthologous sequences of SfAPN4 were identified through similarity search in the non redundant database (nr) of the National Center for Biotechnology Information (NCBI) in the United States. The results are shown in FIG. 5. These orthologous proteins exist in various biological communities, with the vast majority being Lepidoptera pests, for example, sequence alignment with PxAPN4a (Plutella xylostella, GenBank: MG873050), PxAPN4b (Plutella xylostella, GenBank: MG873051), CmAPN4 (Cnaphalocrocis medinalis, GenBank: ADZ05468), CsAPN4 (Chilo suppressalis, GenBank: ADZ57273), HaAPN4 (Helicoverpa armigera, GenBank: AAP37950), HpAPN4 (Helicoverpa punctigera, GenBank: AAF37559), OfAPN4 (Ostrinia furnacalis, GenBank: ACB87202), OnAPN4 (Ostrinia nubilalis, GenBank: ACV74256), SeAPN4 (Spodoptera exigua, GenBank: AAP44967), SlituAPN4 (Spodoptera litura, GenBank: AAK69605) showed highly homologous, which may indicate that they can both be recognized as specific targets by NGR-JAZ and plant derived transcriptional repressor proteins comprising a NGR, as well as their derived peptides and modified products. Additionally, it suggests that they are evolutionarily and functionally related to SfAPN4 and are considered to have similar modes of action.

### Example 2: Applications of NGR-GhJAZ Protein in Killing Fall Armyworms and Cotton Bollworms on Grasslands

### 1. Expression and Purification of GhJAZ8 and GhJAZ8^{LGK} Proteins

PET-30a-GhJAZ8: a vector obtained by inserting the coding gene DNA sequence of GhJAZ8 shown in SEQ ID No. 4 between NdeI and HindIII of a pET-30a (+) vector (Unibio Biotech, catalog number: VT1212), with the GhJAZ8 gene fused with a 6× his tag (located after the GhJAZ8 promoter ATG) to express a GhJAZ8 fusion protein;
PET-30a-GhJAZ8^{LGK}: a vector obtained by inserting the coding gene DNA sequence of GhJAZ8^{LGK} (N43G44R45 mutated to L43G44K45) shown in SEQ ID No. 25 between NdeI and HindIII of a pET-30a (+) vector (Unibio Biotech, catalog number: VT1212), with GhJAZ8^{LGK} gene fused with a 6× his tag (located after the GhJAZ8^{LGK} promoter ATG) to express a GhJAZ8^{LGK} fusion protein;
transform the above pET-30a-GhJAZ8 and pET-30a-GhJAZ8LGK into Escherichia coli BL21, respectively, stand on ice for 30 minutes, heat shock at 42° C. for 60 seconds, and place on ice for 2 minutes. Add LB medium at 37° C., 200 rpm, for 1 hour, coat on LB plates with corresponding antibiotics, and incubate at 37° C. Single clones were picked up after occurrence of the clonal plaques, and cultured with shaking, then the culture was expanded until the medium began to turn slightly white. IPTG (specifically 0.5 mM final concentration) was added for induction and temperature of 37° C. was set for overnight culture. Then the resulted culture was centrifuged at 5000 rpm, 4° C. for 10 minutes, and resuspended in equal volume PBS (with PMSF and DTT added). The suspension was sonicated (sonication time: 20 minutes, interval time: 5 seconds, sonication power: 200 W) for 5 seconds, and placed on ice for 5 seconds, until the resuspended solution becomes significantly clear. A centrifugation at 13000 rpm, 4° C., for 10 minutes was conducted to separate the supernatant and inclusion bodies.

The supernatant was transferred into a protein purification column (Genscript Biotech, catalog number: L00250-25; elution with low pH buffer and imidazole solution, the eluent was collected as a target protein), washed and eluted after being incubated, and detected using a protein pre-staining solution drop by drop and SDS-PAGE, and the protein was collected.

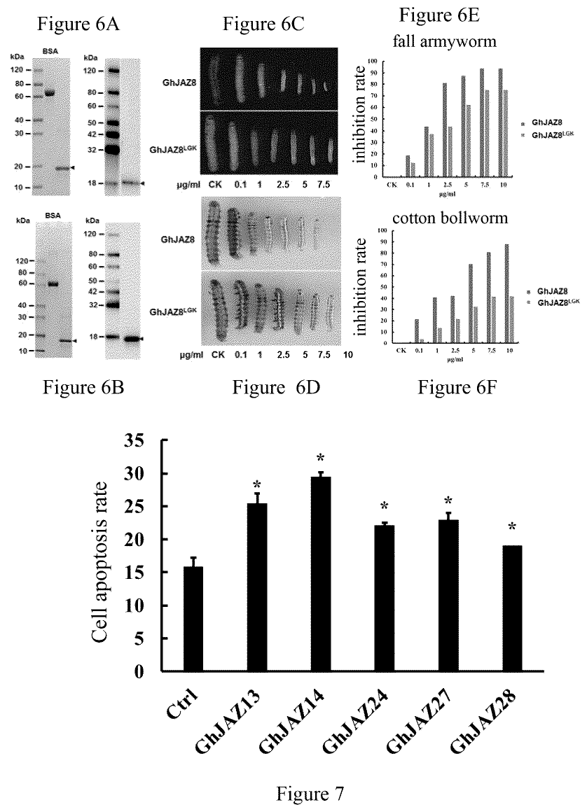

As shown in FIGS. 6A and 6B, a GhJAZ8 fusion protein (N-terminal fused His tag) at a concentration of 2.39 mg/ml and a GhJAZ8^{LGK} fusion protein (N-terminal fused His tag) at a concentration of 1.99 mg/ml were obtained.

### 2. Killing Insects

Treatment group: add the prepared insect feed (Henan Jiyuan Baiyun Industrial Co., Ltd., product name: Grassland armyworm artificial feed) to a 24 well plate, with a volume of 200 microliters per well; use a film covering method to add diluted GhJAZ8 protein solutions of different concentrations (diluted with PBS) to the surface of the feed, and shake in the incubator until the protein is immersed in the culture medium. Take grassland armyworm and cotton bollworm larvae that have not been fed within the first 12 hours of incubation as experimental subjects, and place them in a culture box, with each concentration of protein applied to 100 larvae.

Set 16 replicates for each protein concentration and 3 parallel measurement groups for each dose.

Control group: different from the experimental group, PBS solution was added to the surface of the feed using a film covering method, and the rest were the same.

After 7 days, age and weight will be recorded for statistical analysis.

$$Inhibition rate = ( 1 - average individual weight of larvae in the treatment group \text{} / average individual weight of larvae in the control group ) * 100$$

The results are shown in FIGS. 6C, 6D, 6E, and 6F. The results indicate that after treatment with GhJAZ8 protein, major agricultural pests such as fall armyworm and cotton bollworm showed delayed phenotypic growth and excellent inhibitory activity; however, the inhibitory effect of GhJAZ8^{LGK} protein with mutated “NGR” domain on the growth of fall armyworm and cotton bollworm was weakened.

In order to further investigate the insect resistance effects of five other NGR GhJAZ proteins, GhJAZ13, GhJAZ14, GhJAZ24, GhJAZ27, and GhJAZ28, the following experiments were conducted:

Experimental group: dissolve 5 NGR-GhJAZ proteins in PBS solution, respectively, to obtain protein solutions with a concentration of 0.5 M. Then add different protein solutions to Sf9 cell culture media (5*10⁴ cells/ml) (5 microliters of protein solution per 100 microliters of cell culture medium), culture for 24 hours, and detect apoptosis.

Control group: unlike the experimental group, PBS was added to Sf9 cell culture medium as a control.

The detection method is as follows: digest the adherent cells with trypsin, collect them in a centrifuge tube, centrifuge and precipitate the cells, remove the supernatant, wash once with PBS, and use a cell apoptosis detection kit (Wuhan Procell Life Technology Co., Ltd., catalog number: P-CA-201) for detection of cell apoptosis by flow cytometry.

As shown in FIG. 7, compared with the control group (ctrl), Sf9 cells treated with 5 NGR GhJAZ proteins had an apoptosis rate significantly higher than that of the control group, indicating that all 5 NGR GhJAZ proteins are toxic to fall armyworm cells.

### Example 3: GhJAZ8 Inhibits Cell Cycle by Interacting with SfHDAC3

Based on the existing Spodoptera frugiperda transcriptome and genome databases in combination with the NCBI genome database, PCR amplification was performed via designed primers (template: maize type Spodoptera frugiperda larvae whole tissue cDNA, SfHDAC3-F: 5′-ATGCTTCTCGGTGACATCGAGC-3′ (SEQ ID NO: 45), SfHDAC3-R: 5′-CTAAGGGTCCTTGTTCTCAACC-3′ (SEQ ID NO: 46)) to obtain the full-length sequence of SfHDAC3 (amino acid sequence as shown in SEQ ID NO: 23, nucleotide sequence as shown in SEQ ID NO: 24). Previous studies have shown that RNAi mediated knockdown of HDAC3 in a human colon cancer cell line or HeLa cell led to accumulation of G2 and/or M phase cells, loss of H3Ser10 phosphorylation in the mid stage histone, and mitotic breakdown.

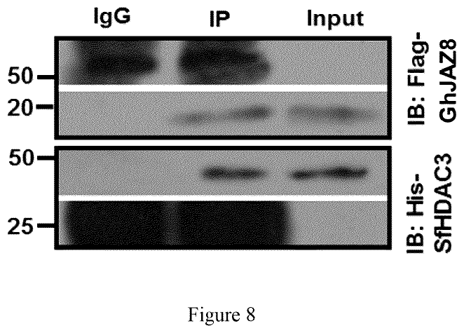

Furthermore, in vivo interaction between GhJAZ8 and SfHDAC3 was validated through CoIP experiments (FIG. 8, using the same experimental method as in III of Example 1).

Therefore, it may indicate that after JAZ protein and its derivatives (including polypeptides) and modified products based on the above substances entered cells via a cell surface receptor, APN4, they inhibited HDAC3 transcription, arrested the cell cycle in G2 and/or M phases, caused mitotic breakdown, and thus produced toxicity to pest cells.

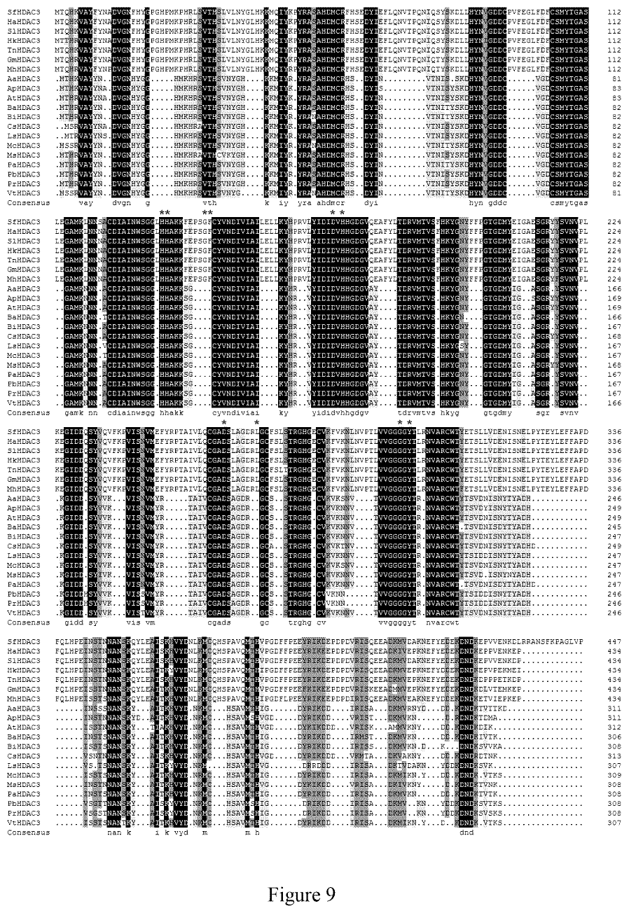

Using BLAST and PSI-BLAST, the orthologous sequences of SfHDAC3 were identified through similarity search in the non redundant database (nr) of the National Center for Biotechnology Information (NCBI) in the United States. These orthologous proteins exist in various biological communities, with the vast majority being Lepidoptera pests, for example, the results of sequence comparison with SlHDAC3 (GenBank: XP_022831573.1, Spodoptera litura), HaHDAC3 (GenBank: XP_021195587.1, Helicoverpa armigera), TnHDAC3 (GenBank: XP_026740888.1, Trichoplusia ni), HkHDAC3 (GenBank: XP_026333196.1, Hyposmocoma kahamanoa), GmHDAC3 (GenBank: XP_026754881.1, Galleria mellonella), MhHDAC3 (GenBank: XP_034827651.1, Maniola hyperantus), AaHDAC3 (GenBank: XP_041972205.1, Aricia agestis), MsHDAC3 (GenBank: KAG6455343.1, Manduca sexta), BiHDAC3 (GenBank: CAH0713927.1, Brenthis ino), ApHDAC3 (GenBank: CAB3219866.1, Arctia plantaginis), CsHDAC3 (GenBank: RVE44174.1, Chilo suppressalis), PaHDAC3 (GenBank: XP_039752448.1, Pararge aegeria), VtHDAC3 (GenBank: XP_026489054.1, Vanessa tameamea), BaHDAC3 (GenBank: XP_023947594.1, Bicyclus anynana), AtHDAC3 (GenBank: XP_013193311.1, Amyelois transitella), McHDAC3 (GenBank: XP_045454150.1, Melitaea cinxia), LsHDAC3 (GenBank: VVD00622.1, Leptidea sinapis), PrHDAC3 (GenBank: XP_022129510.1, Pieris rapae), PbHDAC3 (GenBank: XP_045518296.1, Pieris brassicae) showed highly conserved (FIG. 9), possibly indicating that they can both be recognized as specific targets by NGR-JAZ and plant derived transcriptional repressor proteins comprising a NGR, as well as their derived peptides and modified products. Additionally, it suggests that they are evolutionarily and functionally related to SfAPN3 and are considered to have similar modes of action.

### Example 4: Preparation of GhJAZ8 Transgenic Plant and Identification of its Insect Resistance

### 1. Construction of GhJAZ8 Plants

The plant expression vector is a pMDC100 vector (BioVector, catalog number: CD3-746), which contains one set of CaMV35S promoter to control the plant expression elements of NPTII gene, one set of GhJAZ8 self-promoter and terminator to control the plant expression elements of target gene GhJAZ8.

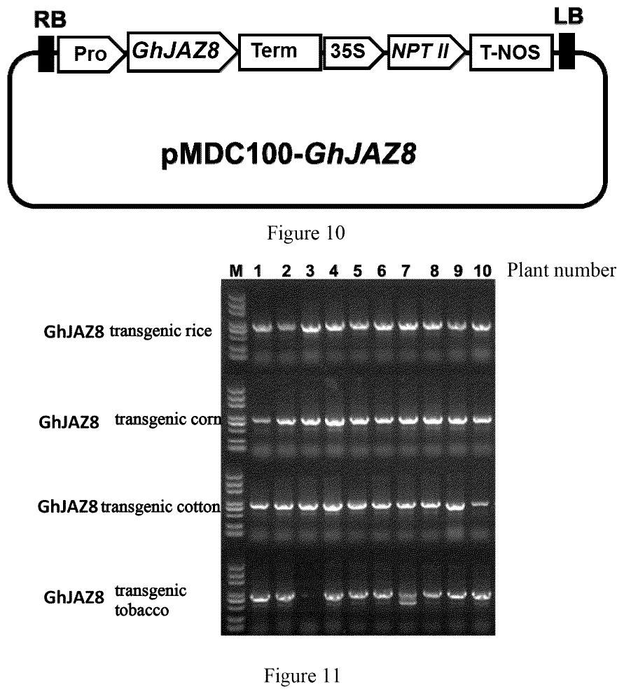

pMDC100-GhJAZ8 (as shown in FIG. 10) is a vector obtained by inserting a GhJAZ8 gene shown in SEQ ID No. 4 between attR1 and attR2 sites of a pMDC100 vector. Pro: promoter of GhJAZ8 (SEQ ID NO: 19); Term: terminator of GhJAZ8 (SEQ ID NO: 20). 35S: plant constitutive promoter derived from cauliflower mosaic virus CaMV (SEQ ID NO: 26); NPTII represents the neomycin phosphotransferase gene, which has resistance to kanamycin (SEQ ID NO: 27); T-NOS: Nos terminator (SEQ ID NO: 28); LB: left boundary of T-DNA; RB: right boundary of T-DNA; the plant expression vector is a pMDC100 vector.

Then, pMDC100-GhJAZ8 was introduced into an Agrobacterium strain LBA4404 using electrical stimulation to obtain a recombinant Agrobacterium.

Then, the recombinant Agrobacterium was transformed into rice using a hypocotyl transformation method (recipient variety: Nipponbare, transformation method reference: Zhao W, Zheng S, Ling H Q. An efficient regeneration system and Agrobacterium-mediated transformation of Chinese upland rice cultivar Handao297. Plant Cell Tissue & Organ Culture. 2011, 106(3):475.), Corn (receptor variety: B73, transformation method literature: Lee H, Zhang Z J. Agrobacterium-mediated transformation of maize (Zea mays) immature embryos. Methods Mol Biol. 2014; 1099:273-280.), Cotton (recipient variety: ZM24, transformation method literature: Yang Z, Ge X, Yang Z, Qin W, Sun G, Wang Z, Li Z, Liu J, Wu J, Wang Y, Lu L, Wang P, Mo H, Zhang X, Li F. Extensive intraspecific gene order and gene structural variations in upland cotton cultivars. Nat Commun. 2019 Jul. 5; 10(1):2989. doi: 10.1038/s41467-019-10820-x. PMID: 31278252; PMCID: PMC6611876.), and Tobacco (recipient variety: Nicotiana benthamiana, transformation method literature: Sunilkumar G, Vijayachandra K, Veluthambi K. Preincubation of cut tobacco leaf explants promotes Agrobacterium-mediated transformation by increasing vir gene induction. Plant Science, 1999. 141(1):51-58.): successively cultivating sterile seedlings, preparing explants, immersing plant explants in Agrobacterium solution, then dedifferentiating to form resistant callus tissue, regenerating into seedlings, and finally obtaining resistant plants, obtaining TO generation of transgenic GhJAZ8rice, TO generation of transgenic GhJAZ8 corn, TO generation of transgenic GhJAZ8cotton, and TO generation of transgenic GhJAZ8 tobacco.

### 2. Molecular Identification

After the TO generation of transgenic GhJAZ8rice (GhJAZ8 transgenic rice), TO generation of transgenic GhJAZ8corn (GhJAZ8 transgenic corn), TO generation of transgenic GhJAZ8 cotton (GhJAZ8 transgenic cotton), and TO generation of transgenic GhJAZ8 tobacco (GhJAZ8 transgenic tobacco) were transplanted and grow to the vegetative growth stage, 0.5 g of leaves were taken to extract genomic DNA, and PCR amplification was performed using cross vector primers (GhJAZ8-F: ggtttacccgccaatatatcc (SEQ ID NO: 47), GhJAZ8-R: tcaattcgaacatggctataac (SEQ ID NO: 48).

The amplification products were detected, and the results were shown in FIG. 11. M was the DNA marker, which was 5000, 3000, 2000, 1000, 750, 500, 250, and 100 bp from top to bottom. The primer used was a cross vector primer, and the product size of 967 bp obtained indicated a positive transgenic plant, indicating that the T-DNA segment of the plant expression vector had been integrated into the genomes of transgenic rice, corn, cotton, and tobacco, and the transgenic plants have been successfully constructed.

### 3. Insect Resistance Identification

Cultivate T0 generation of positive transgenic materials to T3 generation of transgenic GhJAZ8 rice, T1 generation of transgenic GhJAZ8 corn, T2 generation of transgenic GhJAZ8 cotton, and T3 generation of transgenic GhJAZ8 tobacco, respectively.

### 1) Transgenic GhJAZ8 Rice

T3 generation of transgenic GhJAZ8 rice and wild-type rice (variety name: Nipponbare) seedlings with consistent growth (about 1 month old) in a whole pot, 50 larvae of the newly hatched fall armyworm were introduced into each pot, and 3 replicates were set up. The seedlings were placed in an artificial greenhouse and photographed on the 7th day after inoculation.

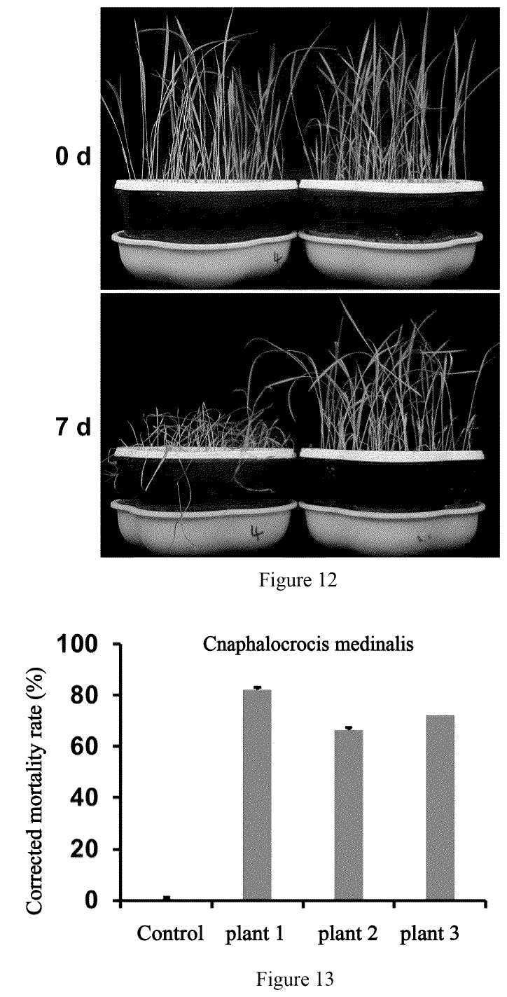

The phenotypic differences between GhJAZ8 transgenic rice and wild-type rice on day 7 after being fed by the fall armyworm are significant, as shown in FIG. 12. The wild-type rice was on the left, and the T3 generation of GhJAZ8 transgenic rice was on the right. The wild-type rice fed by the fall armyworm died in the whole pot, and the larvae had grown to the 3rd instar with good development; in contrast, the growth of T3 generation of transgenic GhJAZ8 rice plants was not significantly affected, and no live insects were observed on the plants of T3 generation of transgenic GhJAZ8 rice.

Using the indoor detached leaf inoculation method a sufficient amount of leaves from different strains (strain 1, strain 2, and strain 3) of T3 generation of yong and tender transgenic GhJAZ8 rice plants (about 1 month old) were taken and placed in large culture dishes. 30 newly hatched Cnaphalocrocis medinalis larvae were introduced into each dish, with 10 replicates set up, placed in an artificial climate chamber and replaced with fresh leaves of the corresponding growth stage the next day. The survival of the larvae was recorded on the 7th day of insect inoculation, and the mortality rate of the larvae was calculated. Corrected mortality rate %=(mortality rate of test insects feeding on GhJAZ8 transgenic material %−mortality rate of test insects feeding on wild-type control %)/(1−mortality rate of test insects feeding wild-type control %)×100.

As shown in FIG. 13, control: corrected mortality rate of Cnaphalocrocis medinalis after feeding on wild-type rice; plants 1-3 are different plant numbers of T3 generation of transgenic GhJAZ8 rice. After feeding on GhJAZ8 transgenic rice, the corrected mortality rate of Cnaphalocrocis medinalis reached a high resistance level. Plant 1 had a corrected mortality rate of 82.33% against Cnaphalocrocis medinalis.

### 2) Transgenic GhJAZ8 Cotton

T2 generation of transgenic GhJAZ8 cotton (OE) and wild-type cotton (WT, variety name: ZM24) seedlings with consistent growth in a whole pot, 50 larvae of the newly hatched fall armyworm were introduced into each pot, and 3 replicates were set up, placed in an artificial greenhouse and on the 7th day of inoculation, samples were taken from the larvae and their intestines were dissected. Midgut tissue slices were prepared and the midgut tissue structure of cotton bollworm with TUNNEL-IF. Green fluorescence marks dead cells, blue fluorescence indicates nuclear signal, and green fluorescence indicates positive staining of dead cells.

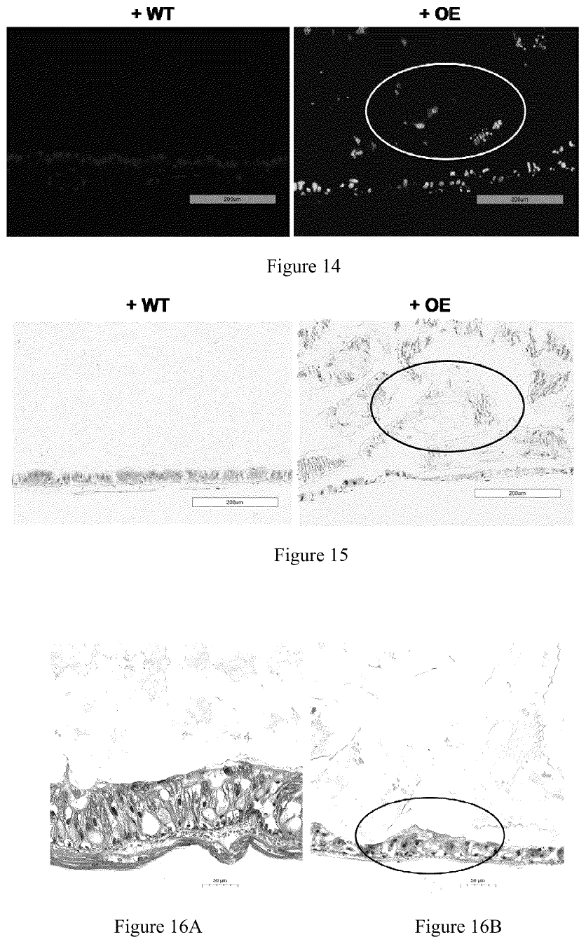

As shown in FIG. 14, the phenotype of midgut cells in cotton bollworms feeding on wild-type cotton leaves was normal, while the midgut cells in cotton bollworms feeding on GhJAZ8 transgenic cotton died in large numbers, and the green fluorescence signal of the dead cells (in the circle on the right) was strong.

### 3) Transgenic GhJAZ8 Corn

T1 generation of transgenic GhJAZ8 corn (OE) and wild-type corn (WT, variety name: B73) seedlings with consistent growth in a whole pot, 50 larvae of the newly hatched fall armyworm were introduced into each pot, with 3 replicates set up, placed in an artificial greenhouse and on the 7th day of inoculation, samples were taken from the larvae and their intestinal tract were dissected. Method as that in transgenic GhJAZ8 cotton, the midgut tissue structure of fall armyworm was stained with TUNNEL. Tunel, TdT mediated dUTP nick end labeling (deoxyribonucleotide end transferase mediated dUTP nick end labeling technique). Tunnel bright field staining: violet indicates nuclear staining, while brown staining indicates positive staining (cell apoptosis).

As shown in FIG. 15, the midgut cells of the fall armyworm fed on T1 generation of transgenic GhJAZ8 corn leaves also exhibited a large number of dead phenotypes (within the circle on the right).

### 4) Transgenic GhJAZ8 Tobacco

T3 generation of transgenic GhJAZ8 tobacco (OE) and wild-type tobacco (WT, variety name: Nicotiana benthamiana) seedlings with consistent growth in a whole pot, 50 larvae of the newly hatched fall armyworm were introduced into each pot, with 3 replicates set up, placed in an artificial greenhouse and on the 7th day of inoculation, samples were taken from the larvae and their intestinal tract were dissected. The midgut tissue structure of the fall armyworm was stained with HE (40×). HE staining refers to hematoxylin eosin staining; Hematoxylin stains the cell nucleus, and the staining itself is blue; the cytoplasm is stained with eosin, resulting in a purple red color.

As shown in FIGS. 16A and 16B, FIG. 16 A represents the phenotype of the midgut tissue from the fall armyworm after feeding on wild-type tobacco leaves, while FIG. 16B represents the phenotype of the midgut tissue from the fall armyworm after feeding on T3 generation of transgenic GhJAZ8 tobacco leaves; it was found in the midgut tissue slices of the fall armyworm larvae after feeding on T3 generation of transgenic GhJAZ8 tobacco, the cell layer of the midgut wall of the fall armyworm larvae after feeding on T3 generation of transgenic GhJAZ8 tobacco are significantly degenerative, with columnar cells shedding and enlarging, goblet cells lysed, and intercellular gaps appearing (in the circle on the right).

In sum, it was indicated that GhJAZ8 transgenic rice, corn, cotton, and tobacco have good resistance to various Lepidoptera pests such as fall armyworm, cotton bollworm, and Cnaphalocrocis medinalis.

### INDUSTRIAL APPLICATION

The present invention is the first to express the representative GhJAZ5, GhJAZ8, and GhJAZ16 of 30 JAZ (JASMONATE ZIM DOMAIN) proteins (Sun H, Chen L, Li J, Hu M, Ullah A, He X, Yang X, Zhang X. The JASMONATE ZIM-Domain Gene Family Mediates JA Signaling and Stress Response in Cotton. Plant Cell Physiol. 2017 Dec. 1; 58(12):2139-2154. doi: 10.1093/pcp/pcx148. PMID: 29036515.), which showed certain insecticidal effects, with the best effect for GhJAZ8. Further analysis revealed that GhJAZ8 comprises an NGR (i.e., Asn-Gly-Arg amino acids) domain, which is a domain that is widely used in medicine and can specifically target to a receptor protein APN (aminopeptidase N). JAZ is a typical plant derived transcriptional repressor protein, and NGR-JAZ refers to a plant derived JAZ protein containing a NGR domain. The NGR domain was discovered for the first time in plant JAZ. Further analysis revealed that NGR-JAZ, represented by GhJAZ8, comprises six members in the cotton genome. The NGR-JAZ protein can poison major agricultural pests such as the fall armyworm, which is the first discovery. Through toxicity experiments on fall armyworm cell lines and feeding experiments on live larvae, the results showed that NGR-JAZ protein had significant toxic effects on them. Through yeast two hybrid Y2H and co immunoprecipitation CoIP experiments, it was found that NGR targets to a cell surface receptor APN of the fall armyworm and enters insect cells, interacting with a key cell cycle protein, SfHDAC3, to disrupt mitosis, thereby exerting a toxic killing effect on pests. Big data analysis also found that APN is highly conserved among major agricultural pests such as cotton bollworms (Lepidoptera), fleahoppers (Hemiptera, Apolygus lucorum (Meyer-Dûr.),), and aphids (Homoptera, cotton aphids). Through live feeding experiments on cotton bollworms, it was found that NGR-JAZ also has toxic killing effects on them. Based on this, it can be inferred that NGR-JAZ and its derivatives (comprising polypeptides) also have toxic killing effects on major agricultural pests such as fleahoppers and aphids. To this end, a GhJAZ8 overexpression vector was created and transgenic rice, corn, cotton, and tobacco were obtained, all of which showed good resistance to fall armyworm and various Lepidoptera pests.

In summary, the present invention discovered for the first time that a JAZ protein, especially NGR-JAZ and derived polypeptides thereof, have broad-spectrum insect resistance, for which no relevant reports are found yet all over the world, and it has significant application value in major agricultural pest control.

1. Use of a GhJAZ protein comprising a NGR domain or a coding nucleic acid thereof, or a recombinant vector, expression cassette or recombinant bacterium comprising the coding nucleic acid, or a substance having the GhJAZ protein as an active ingredient in at least one of:
1) insecticidal;
2) insect resistance;
3) preparation of an insecticidal or insect resistant product;
4) improvement of plant insect resistance;
5) cultivation of an insect resistant plant;
6) prevention and control of a plant disease and a pest;
the NGR domain has an amino acid sequence of SEQ ID NO: 17.

2. The use according to claim 1, characterized in that:
the GhJAZ protein comprising the NGR domain is derived from Gossypium, Tripterygium wilfordii, Hibiscus syriacus, Durio zibethinus, Herrania umbratica, Theobroma cacao, Nelumbo nucifera, or durian.

3. The use according to claim 1, characterized in that:
the GhJAZ protein comprising the NGR domain is any of:
1) a protein shown in SEQ ID NO: 5, SEQ ID NO: 7, SEQ ID NO: 9, SEQ ID NO: 11, SEQ ID NO: 13, or SEQ ID NO: 15;
2) a protein derived from A by adding to an end of the amino acid sequence of the protein shown in 1) a tag sequence and having the same activity;
3) the protein is a protein that has at least 95%, 98%, and 99% sequence identity with that of 1) and comprises a polypeptide shown in SEQ ID NO: 17.

4. The use according to claim 1, characterized in that:
the coding nucleic acid is a DNA molecule of any of 1)-3):
1) a DNA molecule having a coding region shown in SEQ ID NO: 6, SEQ ID NO: 8, SEQ ID NO: 10, SEQ ID NO: 12, SEQ ID NO: 14, or SEQ ID NO: 16;
2) a DNA molecule that is capable of hybridizing with the defined DNA sequence shown in 1) under a harsh condition and encodes a protein having the same function;
3) a DNA sequence that has at least 70%, at least 75%, at least 80%, at least 85%, at least 90%, at least 95%, at least 96%, at least 97%, at least 98%, or at least 99% homology with the defined DNA sequence shown in 1) and encodes a protein having the same function.

5. The use according to claim 1, characterized in that the insect is a plant pest.

6. The use according to claim 5, characterized in that the plant pest is a Lepidoptera pest.

7. The use according to claim 1, characterized in that:
the substance having the GhJAZ protein as an active ingredient is a fusion protein formed by fusing the GhJAZ protein with other protein and having the GhJAZ protein as the active ingredient, a mixture composed of the GhJAZ protein and other component and having the GhJAZ protein as the active ingredient.

8. A method for cultivating an insect resistant transgenic plant, which is 1) or 2):
1) the method comprises the steps of: increasing the content and/or activity of a GhJAZ protein comprising a NGR domain in a target plant to obtain a transgenic plant;
2) the method comprises the steps of: increasing the expression of a nucleic acid molecule encoding a GhJAZ protein comprising a NGR domain in a target plant to obtain a transgenic plant;
the NGR domain has an amino acid sequence of SEQ ID NO: 17;
the transgenic plant has an insect resistance higher than that of the target plant.

9. The method according to claim 8, characterized in that the insect is a plant pest.

10. The method according to claim 9, characterized in that the plant pest is a Lepidoptera pest.

11. The method according to claim 8, characterized in that:
the GhJAZ protein comprising the NGR domain is derived from Gossypium, Tripterygium wilfordii, Hibiscus syriacus, Durio zibethinus, Herrania umbratica, Theobroma cacao, Nelumbo nucifera, or durian.

12. The method according to claim 8, characterized in that:
the GhJAZ protein comprising the NGR domain is any of:
1) a protein shown in SEQ ID NO: 5, SEQ ID NO: 7, SEQ ID NO: 9, SEQ ID NO: 11, SEQ ID NO: 13, or SEQ ID NO: 15;
2) a protein derived from A by adding to an end of the amino acid sequence of the protein shown in 1) a tag sequence and having the same activity;
3) the protein is a protein that has at least 95%, 98%, and 99% sequence identity with that of 1) and comprises a polypeptide shown in SEQ ID NO: 17.

13. The method according to claim 8, characterized in that:
the nucleic acid molecule encoding the GhJAZ protein comprising the NGR domain is a DNA molecule of any of 1)-3):
1) a DNA molecule having a coding region shown in SEQ ID NO: 6, SEQ ID NO: 8, SEQ ID NO: 10, SEQ ID NO: 12, SEQ ID NO: 14, or SEQ ID NO: 16;
2) a DNA molecule that is capable of hybridizing with the defined DNA sequence shown in 1) under a harsh condition and encodes a protein having the same function;
3) a DNA sequence that has at least 70%, at least 75%, at least 80%, at least 85%, at least 90%, at least 95%, at least 96%, at least 97%, at least 98%, or at least 99% homology with the defined DNA sequence shown in 1) and encodes a protein having the same function.

14. A method for killing insects or resisting insects, comprising the steps of: applying a GhJAZ protein comprising a NGR domain to the insects, to achieve insecticidal or insect resistance.

15. The method according to claim 14, characterized in that the insect is a plant pest.

16. The method according to claim 15, characterized in that the plant pest is a Lepidoptera pest.

17. The method according to claim 14, characterized in that:
the GhJAZ protein comprising the NGR domain is derived from Gossypium, Tripterygium wilfordii, Hibiscus syriacus, Durio zibethinus, Herrania umbratica, Theobroma cacao, Nelumbo nucifera, or durian.

18. The method according to claim 14, characterized in that:
the GhJAZ protein comprising the NGR domain is any of:
1) a protein shown in SEQ ID NO: 5, SEQ ID NO: 7, SEQ ID NO: 9, SEQ ID NO: 11, SEQ ID NO: 13, or SEQ ID NO: 15;
2) a protein derived from A by adding to an end of the amino acid sequence of the protein shown in 1) a tag sequence and having the same activity;
3) the protein is a protein having at least 95%, 98%, and 99% sequence identity with that of 1) and comprising a polypeptide shown in SEQ ID NO: 17.

19. Use of a substance that inhibits the expression of a NGR domain in a GhJAZ protein comprising the NGR domain in reducing insecticidal or insect resistance of the GhJAZ proteins comprising the NGR domain;
or, use of a substance that mutates a NGR domains in a GhJAZ protein comprising the NGR domain in reducing the insecticidal or insect resistance of the GhJAZ protein containing the NGR domain.


SEQUENCE LISTING (48 sequences)

SEQ ID NO: 1 - protein, 240 aa, Artificial sequence
  Synthesized
MSSCSESTAMKPARSPEKPSFAQTCNLLSQYLKEKGSFGDLSLGMTCNVEANGTPVVPPPTMNLFPLNDKSDDVCGRNGGNPKNLTSMDLFPQQAGLASKDDSPNKLEPQTAPMTIFYGGQVIVFNDFPANKAKEIMLLASNSSSQSNNSFNPIPFTSSIARSPIESSIGVPPTSKPVHPAQRAVPGDLPIARRASLHRFLEKRKDRITAKAPYQINNSAAAPSLSGDNKSWLGLAAQSP

SEQ ID NO: 2 - DNA, 723 bp, Artificial sequence
  Synthesized
atgtcgtcttgctcggaatctacggctatgaaaccggctaggtcaccggagaagccaagttttgcccaaacttgtaatttgttgagccagtacttgaaggagaaaggtagctttggggatctatctctgggaatgacatgcaacgttgaagctaatgggacaccagtggtgccgcctccgaccatgaatctgttccctctcaatgacaagtccgatgatgtttgtggccgaaacggtgggaatcctaagaacttgacatccatggatttgttcccccaacaagctggtttggcttctaaagatgatagtcccaacaagttggagcctcagactgcaccaatgaccatcttttatggtggacaagtgattgtgttcaatgattttccagcaaacaaagctaaggaaatcatgcttttagctagcaacagcagctcacaaagcaacaacagcttcaaccccatcccttttacttctagcatagccagaagtccaatcgaatcaagcattggagttcctcctacctcaaaaccggttcaccctgctcagcgagctgtccctggcgatctgccaattgcaaggagagcttcactgcatcggttcctagagaagcgaaaggataggatcactgcaaaagcgccataccagataaataactcagctgcagctccttccctgtccggcgataacaagtcatggctcggtttggctgctcaatcaccatag

SEQ ID NO: 3 - protein, 119 aa, Artificial sequence
  Synthesized
MRRNCNLELRLLPSSYPSDSHDMMEERIESPKTQQQQLTIFYNGRVCVSDVTELQAKAILMLANRERDERMKSPTGWEPVSPTLKSQVKCPNTALSMKRSLQRFLQKRKTRIQATSPYH

SEQ ID NO: 4 - DNA, 360 bp, Artificial sequence
  Synthesized
atgagacgaaactgcaacttggaacttcgtcttcttccttctagttatcccagcgacagtcacgacatgatggaagaaagaatcgaaagcccaaaaacccagcagcaacagctaacaattttctacaatggaagagtttgcgtatctgatgttacagagcttcaggcaaaagccattctaatgttagcaaaccgggaaagagatgaaagaatgaaaagtccaacgggatgggaaccggtgtcgccgacgttaaaatctcaggtgaaatgcccaaacacagcgctttccatgaagagatcgcttcaacggttcctccagaaacgaaagacgcgaatccaagctacctctccttaccattaa

SEQ ID NO: 5 - protein, 370 aa, Artificial sequence
  Synthesized
MERDFIGLRSKNAAITIKVEPSNAQPDDSVLLRGSGMQLSFSNKVSTVPQFLSFDGARDDKPRKATHDTLLSSGFMTIPAADSNKKPHPGLTQKQGGNHYAATTYGLQQLDGHQSRLSHEARIFPSSSQPNHTITVSMNTPLLQPHLASPGQNIIGHTINPQPFTGVPIMAAPVSVVPPSSPIIGTTDLRNAAKSSRAPAQLTIFYAGSVCVYDDVSPDKAQAIMLLAGNVSSTTQSKTAPVTQPQTHIPRPCTLSPFSGLPNHLSVTSHVSLQPVAGSSGTNELTAATRIGALASTNNQPDPPKLVNPAVAVPQARKASLARFLEKRKERVSNTSPYNICKRSESGPLASDGISFSVTSAGSSPLQAIN

SEQ ID NO: 6 - DNA, 1113 bp, Artificial sequence
  Synthesized
atggagagagattttatcggtttgaggtccaaaaatgctgcaattacaatcaaagtagagccttccaatgctcagcctgatgattcagtgttgttgaggggttcagggatgcagttgtctttctcaaacaaggtatctacagttcctcagttcttgtcgtttgatggtgctcgagacgataaaccgagaaaagccacgcatgacactctgttgtcatccggttttatgactattccagctgctgattctaacaagaaaccgcatcctggcttgactcagaaacaagggggaaaccactatgcggctaccacctatggtctccaacaactcgatggacaccaatcccgcctttcccacgaagcaaggatatttccaagttcaagtcagccgaatcacacgattacggtttccatgaacactcctcttctccaaccccatcttgcttcccccggacagaacataatcggtcatactataaacccgcaaccttttaccggagttccaatcatggctgcacctgtatctgttgttcctccatcaagtcctatcatcggtactaccgatcttaggaatgctgccaaatcttctagggcaccagctcaattgactatcttttatgctggatctgtatgtgtgtacgatgatgtatctcccgacaaggcccaagctattatgttacttgctggaaacgtttcttctacgactcaaagtaaaacggcacctgtgactcaaccgcagacacatattccacgaccttgtaccttgtcccctttctcgggtcttccaaatcacctttctgtaacctcccatgtcagtttacagcctgttgcaggatctagtggcaccaatgaactaacagctgcaacaaggataggagctttagcatctactaataaccaaccagatcctccaaaattggttaatcctgcagttgctgtgcctcaggctcgtaaagcatccttggctcgatttttagagaagcgtaaagaaagggtatcaaacacctcaccgtacaacatctgcaaaagatctgaatctggccctcttgcatctgatggcataagtttttccgtgacttctgctggatctagtcctttacaagccatcaattaa

SEQ ID NO: 7 - protein, 125 aa, Artificial sequence
  Synthesized
MRRNCNLELELFPSRMFPGHRQNMVEESKRSPQNQQQQLTIFYNGRVCVCDITELQARAILMRANQETDERIKTPTGSEPDSPTSSSSTSLSRLCSPNAGLSMKKSLQRFLQKRKNRIQATSPYH

SEQ ID NO: 8 - DNA, 378 bp, Artificial sequence
  Synthesized
atgagacgaaactgcaacttggaactggagctttttccttcacgtatgtttccaggccatcgtcaaaacatggtggaagagagcaaaagaagcccacaaaatcaacagcagcagctaacaattttctataatggaagagtttgcgtttgtgatattacagagcttcaggcaagagctattttaatgagggcaaatcaagaaacggatgaaagaataaaaactccaacaggatcggaaccggattcaccgacatcgtcatcatcgacatccctgtctcgattatgtagccctaatgctggtctttccatgaagaaatcacttcaaaggttccttcaaaaaagaaagaacagaatccaagctacgtccccttaccattaa

SEQ ID NO: 9 - protein, 120 aa, Artificial sequence
  Synthesized
MRRNCNLELRLHPSSYSGGDRVEESSENPENQQQQLTIFYNGRVCVCDVTEIQARAILMIANRETDERLRTPRQGSEPASPMVHSQINSPNNGLSTSMKRSLQRFLQKRKNRIQATSPYH

SEQ ID NO: 10 - DNA, 363 bp, Artificial sequence
  Synthesized
atgagacgaaactgcaacttggagcttcgtctccatccttctagttattccggcggcgaccgggtggaagagagcagtgaaaacccagaaaatcaacagcaacagttaacgattttctacaatggaagagtttgtgtttgtgatgttacagagatccaggcaagagccattcttatgattgcaaaccgagaaacggatgaacgactaaggactccgaggcagggatcggaaccggcttcaccgatggtacactctcagataaatagtccgaataatggactttccacaagcatgaagagatcgcttcaacggttcctccaaaaacgaaagaatcgaatccaagctacctctccttaccattaa

SEQ ID NO: 11 - protein, 119 aa, Artificial sequence
  Synthesized
MRRNCNLELRLLPSSYPSDSHDMMEARIESPETQQQQLTIFYNGRVCVSDVTELQAKAILILANRERDERMKSPTGCEPVSPTLKSQVNSPNTSLSMKRSLQRFLQKRKTRIQATSPYH

SEQ ID NO: 12 - DNA, 360 bp, Artificial sequence
  Synthesized
atgagacgaaactgcaacttggaacttcgtcttcttccttctagttatcccagcgacagtcacgacatgatggaagcaagaattgaaagcccagaaacccagcagcaacagctaacaattttctacaatggaagagtttgcgtatctgatgttacagagcttcaggcaaaagccattctaatattagcaaaccgggaaagagatgaaagaatgaaaagtccgacgggatgtgaaccggtgtcgccgacgttaaaatcccaggtgaatagcccaaacacatcactttccatgaagagatcgcttcaacggttcctccagaaacgaaagacgcgaatccaagctacctctccttaccattaa

SEQ ID NO: 13 - protein, 125 aa, Artificial sequence
  Synthesized
MRRKCNLELELFPSRIFPGHRQNMVEESKRIPQNQQQQLTIFYNGRVCVCDVTELQARAILMRANREADERIKTPTGSEPDSPTSSSSTSPSRLCSPNAGLSMKKSLQRFLQKRKNRIQATSPYH

SEQ ID NO: 14 - DNA, 378 bp, Artificial sequence
  Synthesized
atgagacgaaagtgcaacttggaactggagctttttccttcacgtatttttccaggccatcgtcaaaacatggtggaagagagcaaaagaatcccacaaaatcaacagcagcagctaaccattttctataatggaagagtttgcgtttgtgatgttacagagcttcaggcaagagctattttaatgagggcaaatcgagaagcggatgaaagaataaaaactccaacaggatcggaaccggattcaccgacatcgtcatcatcgacatccccgtctcgattatgtagccctaatgctggtctttccatgaagaaatcccttcaaaggttccttcaaaaaagaaagaacagaatccaagctacctccccttaccattaa

SEQ ID NO: 15 - protein, 120 aa, Artificial sequence
  Synthesized
MRRNCNLELRLHPSSYSGGDRVEESSENPENQQQQLTIFYNGRVCVCDVTEIQARAILMIANRETDERLRTPRSGSGPASPTVHSQINSPNNGLSTSMKRSLQRFLQKRKNRIQATSPYH

SEQ ID NO: 16 - DNA, 363 bp, Artificial sequence
  Synthesized
atgaggcgaaactgcaacttggagcttcgtctccatccttctagttattccggcggcgaccgggtggaagagagcagtgaaaacccagaaaatcaacagcaacagttaacgattttctacaatggaagagtttgtgtttgtgatgttacagagatccaggcaagagccattcttatgattgcaaaccgagaaacggatgaacgattaaggactccgaggtcgggatcgggaccggcttcgccgacggtacactctcagataaatagtccgaataatggactttccacaagcatgaagagatcgcttcaacggttcctccaaaaacgaaagaatcgaatccaagctacctctccttaccattaa

SEQ ID NO: 17 - protein, 15 aa, Artificial sequence
  Synthesized
QQQQLTIFYNGRVCV

SEQ ID NO: 18 - DNA, 45 bp, Artificial sequence
  Synthesized
cagcagcaacagctaacaattttctacaatggaagagtttgcgta

SEQ ID NO: 19 - DNA, 1000 bp, Artificial sequence
  Synthesized
ttatttttcgaaaattatttttaatctcactctgaaattaattataaaaataattatgttataagcaaccgactaaaacaaagtttttaaaattgttgtctgtttatcttctacattaattctttagttaatttggtagcttagaaaggctaatattaataacacaacccaaatgtcaaacaaaaaaccatccccaaaagaaaaacccaacccatttctgtgtcatctaattgaagtaatacatcgtttccgtaaatgtcaatacgtctaccattaaaaatttccccccttcacatggccggtattttatttttttatttaattgaattaatgatttaatccagattcgaaaacttcaattaataaaattttgatttaataaaaatattaaaaaataaaaaaaattgatacctttattgattttggattaactgatggagtataaatatacccttttatcctgttactgtaatctaaaatttcattatcaacacatgaaaaggcaatttaattttcatgaaaaaaacatttttgttaagaaaaagtaatgccgacttttaaaaaagcaaatcaggaaaatgaggttatcgattagcatagtagttttattaaaaaggaggaaaaacaggaaaaagtagccgttttgtaggtttgtgaaacaatggaaaaagaagttaggaaataagaagagcgttttccacgtgcttaaattcaaaaaatttagagctgttttgttttattttgatttcttaatgggttgtattgtgtgtcgcgttttttgtgcattgcgtgtttaacatagctttcaatgcccaccccaccacctttcctttatatttaaccccttcttcttcttcttcttcttcttcatcttatcccccaacatagtatcattttcttcttctctttctatatttcatctcttcagcttcttccctaaaccacccaaagtttttaataacagctttagtttaaaacagacttctttttatatataaaa

SEQ ID NO: 20 - DNA, 1000 bp, Artificial sequence
  Synthesized
tatatagaggtcgtcatcaatgcttgaaatttagtggaatttaagcaactgatctatgttgtcggctaggaactattaccctttttatttttttttatttttgttttttgtaaatttatgaatgaataattacgctagtacttgaagagaaagatttttttaaccattaatttgttaatttacgtcaattatgcttaattatttcattgttgtataagtattgttcttgtgatggtctttcatttccatcatttacaaccaatatacaaaaattgtaggcttcatctttcatcttgcttcaaactagtttctttccctttgctagttaaattataatatttttgttcttttagggcttgaatcaaaatgataaaatattttttaatgaatttaaatgttccacgctttacttgaagtaaggaaacaaaacggtcagagtcatttcccctcaacaaaaatatattaaatcattttataatttttgtataatttttttttgtaaaagatgataaaagaaatttatgcataatatttcacaaattaaatattttaaaattgattagaattttctaaataactcttttgtaaataattacgtaaaattcttcttttttttttagttgttaatggattaatattaaaatattaaaagtgttattaactatttaaggtgcgatttaaataatttatatattttttaattacgaatccgcttttataatataaaagatgatgatgtgtgaaacaatgtgacaatctatttgtcacaaaagtaaccatatataccatataaaattgttttcttaaatgtttttgtaccttataatatatgtatatatgagatctaaggttgggttttagtatcctaaaactaaaagctaactttgctacaatttattgtaaattcgtaattcgttcagttacgaaaaattttgaaaatatgtttatttatccataactgtaaaatccactttgggagacaattatatatgcattttaa

SEQ ID NO: 21 - protein, 952 aa, Artificial sequence
  Synthesized
MGTKMLVPAVFCVLLGFAAALLPEDMRSNLEFFDYSSNVHTSAYRLRDNVYPTDMKIDLDNIDLEGATFTGSVEMIVLVRENDLEQISMHQKNLFVTRINVVNNTNGENVQLRSPNPFSYDDKFELLHLHFHLPLVTGSYTITIDYRGRINENPVDRGFYRGYYYYENTRRYYATTQFQPYHARKAFPCFDEPQFKSRYTLSITRPYNLGPSYSNMGISSTQLVGSSIRETFYPTPIISAYLVAFHVSDFVPTNATSTDPRPFNIISRPGVTDQHAYAAEIGVEITNQLDDYFGIEYHNMGQGSVMKNDHIALPDFPSGAMENWGMVNYREAYLLYDPANTNLPNKITIATIMAHELGHKWFGNLVTCFWWSNLWLNESFASFFEYFAAHWADPKLELADQFVVDYVHSALNADASPSATPMNWEEVENNPSITSHFSTTSYAKGASVLRTLEHFVGPRTFRNALRHYLRDNAYGIGTPQLMYRAFEKAINEDHTFLNDFPDVNFEAVFDSWIQNRGSPVVSVDRNVNTGGIVVKQERYQLSGVRPTQTWQIPLSWTEQKHLDFSSTKPKFLLNTSEAALASEEGDNFVIFNIQQSGLYRVKYDDNNWRALSSYLNSNNREQIHKLNRAQIVNDVLYFIRSNDINLELGFHVIDFLRSETDYYVWNGALTQLDWIRRRMEHMPRAHEAFTSYMRGLMNNVINHLGYNEGPNDSTATILNRIQILNYACNIGHSGCISDSLQKWKDFKENNEPVPVNLRRHVYCVGVREGDRSDYDFLFNAYNTSENTADMVIMLRTLPCTKDEDALKHYLQESMYNDRIRIHDRTNAFNFALQGNLENVKIVTQFLRNNYQEIRITYGGEARLILCINALAGFLNTYPEITEFQQWAYMVQVGLGEAFTTSQGVINSAMANLDWGSNNVLEVFKFVSVRSGSPTIFASSFLIIAAMLIQLFR

SEQ ID NO: 22 - DNA, 2859 bp, Artificial sequence
  Synthesized
atgggtaccaaaatgttggttcccgctgtgttttgcgttcttctgggatttgctgctgcacttctcccagaggatatgcggtcaaatttggaattctttgactactcctcaaatgtccatacatcagcttaccgtctccgtgacaatgtgtacccaactgacatgaaaatagatctggataatattgaccttgaaggggctacattcacaggatctgtggagatgatcgttcttgtaagagagaacgatttagagcaaatctctatgcaccagaaaaatctctttgtcaccagaatcaatgttgtcaacaacacaaatggtgaaaatgttcaactgaggtcccccaatcccttctcctatgacgacaaatttgaactcctacaccttcatttccacctacccctcgtcaccggctcctacactatcactatcgactatagaggccgcatcaacgaaaaccctgtcgacagaggtttctacagaggttactattactacgaaaacactcgcaggtactacgccaccacgcagttccagccctaccacgccaggaaagctttcccttgcttcgacgagccacagttcaagtcacgctacacactctccatcactcggccttacaatcttggcccatcttactccaacatgggaatcagttctacccaattggttggcagtagcattcgcgaaacattctaccctactcccatcatttctgcctaccttgtggctttccacgtgagcgacttcgtgcccactaatgccacaagcactgatcctagaccattcaacattatctctcgcccaggagtgacggaccaacatgcatatgctgccgaaatcggtgtggaaataaccaaccagttagatgactattttggcatcgagtaccataatatgggacaagggtcagttatgaagaacgatcatattgctctgcctgactttccgtctggtgctatggaaaactggggaatggtcaactacagagaggcttaccttttatacgaccctgctaacaccaacttaccgaacaaaataaccattgcaaccatcatggctcatgaactgggacacaaatggttcggtaacctggtcacctgtttctggtggagtaacctttggctaaacgaatctttcgctagcttcttcgaatattttgctgcgcattgggctgatccaaaactagaattagctgatcaattcgtcgttgactacgtgcacagtgccctcaatgcagatgccagtccatcggctactcctatgaactgggaagaggttgaaaacaatccctcaatcacttcacactttagtactaccagctatgccaaaggagcttctgtgcttagaacgttggagcattttgttggaccaaggaccttccgtaatgcgctcagacattacttgcgcgacaacgcctacggtatcggtacccctcagctcatgtatcgtgcatttgaaaaggctatcaatgaagatcatacatttttaaatgatttcccggacgtcaactttgaggctgtattcgatagttggatccaaaaccgtggttctccagtcgtgtctgttgaccgtaacgtaaacactggcggtatagttgttaaacaggaacgttaccaactttctggcgttagacccacccaaacttggcagatcccgcttagttggacagagcagaaacatttggacttcagctcgaccaagcctaagtttttgcttaatacatcggaagctgctctggcaagcgaggaaggagacaactttgtcatatttaacattcaacaatctggactgtatcgcgtcaaatacgatgataacaactggagagcactttcatcttacctgaacagcaacaacagggaacaaattcacaaactgaacagagctcaaattgtcaatgatgtgctgtacttcattcgctccaatgacatcaatctggaacttggtttccatgttattgatttcttgaggagtgaaactgattattacgtgtggaacggtgctcttactcaacttgactggatccgtcgtcggatggagcatatgcccagagctcacgaagcgttcactagctacatgcgtggccttatgaacaatgtcatcaaccacctcgggtacaacgaaggccccaacgactctaccgccaccatcctaaacagaatccagatcttaaactacgcttgcaacatcggacacagcggttgcatttctgatagtttacagaaatggaaagatttcaaagaaaacaacgaaccggtacctgtgaaccttcgtcgccatgtctattgcgttggtgttcgtgagggtgatagatctgactacgactttctattcaatgcatacaacacatcagaaaataccgctgatatggtaatcatgcttcgtaccctaccttgcaccaaagatgaagatgctcttaaacattacttgcaagagagcatgtacaacgaccggattcgtatccacgaccgcactaacgctttcaattttgctctgcaaggaaacctagaaaatgtgaaaatcgttacccaattccttagaaataattaccaggaaatcagaataacatatggcggtgaagctcgtctgatcctctgcatcaacgctcttgccggcttcctgaacacgtatcccgaaataacagaattccagcaatgggcctatatggtgcaagtcggcttaggagaggctttcactaccagtcagggtgtaatcaactctgctatggccaatcttgactggggtagcaacaatgtgctagaagtgttcaaattcgtttcagtcagaagtggttctcctactatcttcgcttcatcattcctcatcattgcagctatgctcattcagctattccgttaa

SEQ ID NO: 23 - protein, 447 aa, Artificial sequence
  Synthesized
MTQHKVAYFYNADVGNFHYGPGHPMKPHRLSVTHSLVLNYGLHKKMQIYKPYRASAHDMCRFHSEDYIEFLQNVTPQNIQSYSKDLLHYNVGDDCPVFEGLFDFCSMYTGASLEGAMKLNNSACDIAINWSGGLHHAKKFEPSGSCYVNDIVIAILELLKYHPRVLYIDIDVHHGDGVQEAFYLTDRVMTVSFHKYGNYFFPGTGDMYEIGAESGRYYSVNVPLKEGIDDQSYVQVFKPVISNVMEFYRPTAIVLQCGADSLAGDRLGCFSLSTRGHGECVKFVKNLNVPTLVVGGGGYTLRNVARCWTYETSLLVDENISNELPYTEYLEFFAPDFQLHPEINSTNNANSKQYLEAISKHVYDNLKMCQHSPAVQMTHVPGDFFPEEYRIKDEPDPDVRISQEEADKMVDAKNEFYEDEKDNDKEPVVENKDLRRANSFKPAGLVP

SEQ ID NO: 24 - DNA, 1344 bp, Artificial sequence
  Synthesized
atgactcaacataaagtagcttatttttataacgcggacgtggggaacttccactatgggcccgggcaccccatgaaaccgcatcggttgtcggtgactcatagtttagtgctgaattatggcctgcataaaaagatgcagatatacaaaccgtatagagcgagcgcccatgacatgtgccgcttccacagcgaggattatattgaattcctgcaaaatgtgactccacagaatatacaaagttattcaaaagacttactgcactacaatgtaggtgatgactgtccagtgtttgaagggctgtttgatttctgctcgatgtacacgggggcttcactagagggtgccatgaagctcaacaatagtgcctgtgatattgccatcaactggtctggaggtctacatcatgctaagaagtttgagccttctggttcctgctatgtaaatgacatagtaatagcaatcctagaattactgaaataccacccgagggtgttatacatagacattgacgtccaccacggagatggtgtgcaagaagccttctacctgactgacagagtgatgacggtcagtttccataagtacgggaactacttcttcccaggaactggagacatgtatgaaattggggctgaaagtggaaggtactattctgttaatgtgccgctaaaggaagggattgatgaccagagttatgttcaggttttcaaaccagtaatatctaatgtgatggagttctaccgtccaacggcgatagtgttgcaatgcggcgctgactcgctggcgggtgacagactcgggtgcttctcgctgtccacgcggggccacggagagtgcgtcaagttcgtcaagaacttaaatgtacccactttggttgtgggagggggaggctacacattaaggaacgtggcgcggtgctggacatatgagacgtcgctcctagtcgatgagaatatatcaaatgagttgccctacacggagtatttggagtttttcgcgccagacttccaattgcatccagaaatcaatagcacaaataacgcgaacagcaagcagtatttggaagcgatctcaaaacacgtgtacgacaacctcaagatgtgccagcactcgcctgctgtgcaaatgacgcatgtaccaggcgacttcttccccgaagagtataggataaaggacgagcctgacccagacgtgaggatcagtcaagaggaggcagacaaaatggttgacgctaagaacgagttttatgaggacgaaaaggacaatgataaggaaccagtggttgagaacaaggaccttagaagggccaattcgtttaaacctgcaggactagtcccttag

SEQ ID NO: 25 - DNA, 360 bp, Artificial sequence
  Synthesized
atgagacgaaactgcaacttggaacttcgtcttcttccttctagttatcccagcgacagtcacgacatgatggaagaaagaatcgaaagcccaaaaacccagcagcaacagctaacaattttctacctgggcaaggtttgcgtatctgatgttacagagcttcaggcaaaagccattctaatgttagcaaaccgggaaagagatgaaagaatgaaaagtccaacgggatgggaaccggtgtcgccgacgttaaaatctcaggtgaaatgcccaaacacagcgctttccatgaagagatcgcttcaacggttcctccagaaacgaaagacgcgaatccaagctacctctccttaccattaa

SEQ ID NO: 26 - DNA, 770 bp, Artificial sequence
  Synthesized
cgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctct

SEQ ID NO: 27 - DNA, 795 bp, Artificial sequence
  Synthesized
atggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

SEQ ID NO: 28 - DNA, 318 bp, Artificial sequence
  Synthesized
tgagcgggactctggggttcggatcgatcctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttaca

SEQ ID NO: 29 - protein, 13 aa, Artificial sequence
  Synthesized
QQLTIFYGGRVVV

SEQ ID NO: 30 - protein, 12 aa, Artificial sequence
  Synthesized
QLTIFYGGSVCV

SEQ ID NO: 31 - protein, 13 aa, Artificial sequence
  Synthesized
QRLTIFYNGKMCV

SEQ ID NO: 32 - protein, 15 aa, Artificial sequence
  Synthesized
QQQPLTIFYDGKICV

SEQ ID NO: 33 - protein, 13 aa, Artificial sequence
  Synthesized
QQLTIFYGGRVVV

SEQ ID NO: 34 - protein, 10 aa, Artificial sequence
  Synthesized
QLTIFYSGKV

SEQ ID NO: 35 - protein, 15 aa, Artificial sequence
  Synthesized
EQQQLTIFYDGKVVV

SEQ ID NO: 36 - protein, 4 aa, Artificial sequence
  Synthesized
TIFY

SEQ ID NO: 37 - protein, 5 aa, Artificial sequence
  Synthesized
LTIFY

SEQ ID NO: 38 - protein, 15 aa, Artificial sequence
  Synthesized
QQQPLTIFYDGKICV

SEQ ID NO: 39 - protein, 12 aa, Artificial sequence
  Synthesized
QLTIFYAGSVCV

SEQ ID NO: 40 - protein, 15 aa, Artificial sequence
  Synthesized
QQQQLTIFYNGKVVV

SEQ ID NO: 41 - DNA, 23 bp, Artificial sequence
  Synthesized
cgctggggcaaccatgggtacca

SEQ ID NO: 42 - DNA, 23 bp, Artificial sequence
  Synthesized
gaaatacgagacgacaacgacat

SEQ ID NO: 43 - DNA, 24 bp, Artificial sequence
  Synthesized
gactacaaagacgatgacgacaaa

SEQ ID NO: 44 - DNA, 30 bp, Artificial sequence
  Synthesized
tacccatacgatgttccagattacgcttga

SEQ ID NO: 45 - DNA, 22 bp, Artificial sequence
  Synthesized
atgcttctcggtgacatcgagc

SEQ ID NO: 46 - DNA, 22 bp, Artificial sequence
  Synthesized
ctaagggtccttgttctcaacc

SEQ ID NO: 47 - DNA, 21 bp, Artificial sequence
  Synthesized
ggtttacccgccaatatatcc

SEQ ID NO: 48 - DNA, 22 bp, Artificial sequence
  Synthesized
tcaattcgaacatggctataac